(19) United States
(12) Patent Application Publication
(10) Pub. No.: US 2026/0048003 A1
(43) Pub. Date: Feb. 19, 2026

(54) STABLE CYCLODEXTRIN FREE CARFILZOMIB FORMULATION

(71) Applicant: AMGEN INC., Thousand Oaks, CA (US)

(72) Inventors: Qahera MUNAIM, Thousand Oaks, CA (US); William J. CALLAHAN, III, Thousand Oaks, CA (US); Alona TERAN, Thousand Oaks, CA (US); Sabaha KHAKOO, Thousand Oaks, CA (US)

(21) Appl. No.: 17/810,142

(22) PCT Filed: Jan. 8, 2021
(86) PCT No.: PCT/US2021/012824

§ 371 Date: Jun. 30, 2022

Related U.S. Application Data
(60) Provisional application No. 62/959,833, filed on Jan. 10, 2020.

(51) Int. Cl. A61K 9/00; A61K 9/08; A61K 9/19; A61K 38/07; A61K 47/10; A61K 47/12; A61K 47/18; A61K 47/20; A61K 47/22; A61K 47/26; A61K 47/32

(52) U.S. Cl. CPC A61K 9/0019; A61K 9/08; A61K 9/19; A61K 38/07; A61K 47/10; A61K 47/12; A61K 47/18; A61K 47/20; A61K 47/22; A61K 47/26; A61K 47/32

(57) ABSTRACT

This disclosure provides a stable cyclodextrin free carfilzomib formulation in aqueous solution which is suitable for injection, a kit comprising said cyclodextrin free carfilzomib formulation, and methods for preparation of said cyclodextrin free carfilzomib. Such formulation, kit and methods substantially increase the solubility and stability of the carfilzomib in aqueous solution and facilitate both their manufacture and administration.

### CROSS-REFERENCE TO RELATED APPLICATION

This application claims the benefit of priority of International Application No. PCT/US21/12824, filed Jan. 8, 2021, which claims the benefit of priority of U.S. Provisional Patent Application No. 62/959,833, filed Jan. 10, 2020, which is incorporated by reference in its entirety.

### TECHNICAL FIELD

This disclosure provides a stable cyclodextrin free carfilzomib formulation in aqueous solution which is suitable for injection, a kit comprising said cyclodextrin free carfilzomib formulation, and methods for preparation of said cyclodextrin free carfilzomib. Such formulation, kit and methods substantially increase the solubility and stability of the carfilzomib in aqueous solution and facilitate both their manufacture and administration.

### BACKGROUND

Carfilzomib is a selective proteasome inhibitor approved for the treatment of multiple myeloma. Carfilzomib is a tetrapeptide epoxyketone proteasome inhibitor having the chemical structure:

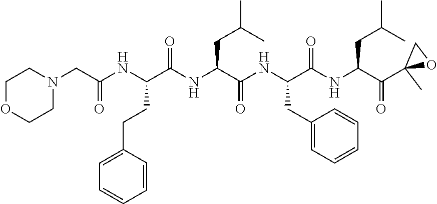 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

that irreversibly binds to the N-terminal threonine-containing active sites of the 20S proteasome, the proteolytic core particle within the 26S proteasome. Carfilzomib has antiproliferative and proapoptotic activities in vitro in solid and hematologic tumor cells. In animals, carfilzomib inhibited proteasome activity in blood and tissue and delayed tumor growth in models of multiple myeloma, hematologic, and solid tumors.

Carfilzomib is commercially marketed under the name Kyprolis® in single dose vials containing 10 mg, 30 mg or 60 mg of the active ingredient. Each vial, in addition to lyophilized carfilzomib, also contains sulfobutylether beta-cyclodextrin, citric acid and sodium hydroxide for pH adjustment (target pH 3.5).

There have been efforts to obtain improved carfilzomib compositions. For instance, substituted cyclodextrin additives have been explored to enhance the solubility of the active ingredient. However, the high cost and limited accessibility of substituted cyclodextrins limits their use in pharmaceutical compositions.

### SUMMARY OF THE INVENTION

Carfilzomib has extremely low aqueous solubility, is pH and concentration sensitive, and has an epoxide ring that is delicate to nucleophilic attack, all in which poses many challenges to prepare stable formulation of carfilzomib without use of cyclodextrins. There remains a need for improved formulations of carfilzomib having improved ease of manufacture, means of administration, and stability over time. There remains a need for formulations which are easy for healthcare providers to prepare and administer. There remains a need for cyclodextrin free carfilzomib formulations having improved stability over time, especially when stored under ambient conditions.

An object of the present invention is to provide a stable, ready-to-use or ready-to-dilute cyclodextrin free carfilzomib formulation.

Another object of the present invention is to provide a kit comprising a stable, ready-to-use or ready-to-dilute, such as lyophilized powder or cake, cyclodextrin free carfilzomib formulation.

Another object of the present invention is to provide a process for preparation of a stable, ready-to-use or ready-to-dilute cyclodextrin free carfilzomib formulation.

Another object of the present invention is to provide a stable, ready-to-use or ready-to-dilute cyclodextrin free carfilzomib formulation which is suitable for injection and wherein the injection is administered intravenously or subcutaneously.

Yet another object of the present invention is to provide methods for treating patients with multiple myeloma by administering the stable ready-to-use or ready-to-dilute cyclodextrin free carfilzomib formulation.

In one embodiment, the present invention provides a cyclodextrin free pharmaceutical composition, comprising:
(i) carfilzomib having the chemical structure:

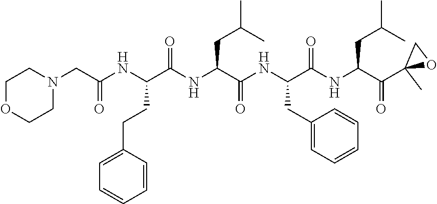 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

or a pharmaceutically acceptable salt thereof;
(ii) a solvent system comprising a pharmaceutically acceptable solvent suitable for injection selected from the group consisting of dimethylsulfoxide (DMSO), N-methyl-2-pyrrolidone (NMP), dimethylacetamide, or ethyl lactate; a co-solvent system selected from C₁₋₄ alkyl alcohol, polyethylene glycol (PEG) optionally in the present of a first co-solubilizing agent; and an aqueous solution having a pH of between 2.5 to 4.5 optionally in the present of a second co-solubilizing agent;
wherein said composition is a ready-to-use injection or is obtained as a lyophilized powder or cake; and wherein the injection is administered intravenously or subcutaneously.

In embodiment 2, the present invention provides the cyclodextrin free pharmaceutical composition according to embodiment 1, wherein the solvent system is dimethylsulfoxide, N-methyl-2-pyrrolidone, or dimethylacetimide.

In embodiment 3, the present invention provides the cyclodextrin free pharmaceutical composition according to any one of embodiments 1 or 2 wherein the co-solvent system is a mixture of ethanol and polyethylene glycol or a mixture of tert-butyl alcohol and polyethyleneglycol optionally in the presence of the first co-solubilizing agent.

In embodiment 4, the present invention provides the cyclodextrin free pharmaceutical composition according to any one of the previous embodiments wherein the co-solvent system is 75% to 92% PEG400:ethanol (1:1, w/w) and in the absence of the first co-solubilizing agent.

In embodiment 5, the present invention provides the cyclodextrin free pharmaceutical composition according to any one of the previous embodiments wherein the co-solvent system is a mixture of ethanol and PEG400 in the presence of the first co-solubilizing agent which is selected from an acid, ester, organic salt, organic base, or a C₁₋₄ alkyl alcohol.

In embodiment 6, the present invention provides the cyclodextrin free pharmaceutical composition according to any one of the previous embodiments wherein the first co-solubilizing agent is an acid or an ester selected from lactic acid, maleic acid, citric acid, benzoic acid, benzene sulfonic acid, acetic acid, or sucrose cocoate.

In embodiment 7, the present invention provides the cyclodextrin free pharmaceutical composition according to any one of the previous embodiments wherein the co-solvent system is an organic salt selected from benzokonium chloride or protamine sulfate.

In embodiment 8, the present invention provides the cyclodextrin free pharmaceutical composition according to any one of the previous embodiments wherein the first co-solubilizing agent is ethanol amine or isopropyl alcohol.

In embodiment 9, the present invention provides the cyclodextrin free pharmaceutical composition according to any one of the previous embodiments wherein the co-solvent system is selected from the group consisting of: 75% to 92% PEG400:ethanol (1:1, w/w); 1.2% to 5% lactic acid in PEG400:ethanol; 1.2% to 5% maleic acid in PEG400:ethanol; 4.6% benzokonium chloride in PEG400:ethanol; 1% to 3.3% protamine sulfate in PEG400:ethanol; 28% to 30% HS Solutol 15 in PEG400:ethanol; 32% sucrose cocoate, PEG400:ethanol; 5% benzoic acid, PEG400:ethanol; 5% benzenesulfonic acid, PEG400:ethanol; 10% isopropyl alcohol, PEG400:ethanol; 1% to 5% citric acid in PEG400:ethanol; 1.2% to 5% acetic acid in PEG400:ethanol; or 5% ethanolamine in PEG400:ethanol.

In embodiment 10, the present invention provides the cyclodextrin free pharmaceutical composition according to any one of the previous embodiments wherein the co-solvent system is 1.2% to 5% lactic acid in PEG400:ethanol.

In embodiment 11, the present invention provides the cyclodextrin free pharmaceutical composition according to any one of the previous embodiments wherein the ratio of lactic acid to the carfilzomib is 1.5:2 by weight.

In embodiment 12, the present invention provides the cyclodextrin free pharmaceutical composition according to any one of the previous embodiments wherein the ratio of lactic acid to the carfilzomib is 0.4:2 by weight.

In embodiment 13, the present invention provides the cyclodextrin free pharmaceutical composition according to any one of the previous embodiments wherein the final maximum lactic acid concentration is 0.15%.

In embodiment 14, the present invention provides the cyclodextrin free pharmaceutical composition according to any one of the previous embodiments wherein the aqueous solution has a pH of between 3.0 to 3.5 and in the absence of the second co-solubilizing agent.

In embodiment 15, the present invention provides the cyclodextrin free pharmaceutical composition according to any one of the previous embodiments wherein the aqueous solution has a pH of between 3.0 to 3.5

In embodiment 16, the present invention provides the cyclodextrin free pharmaceutical composition according to any one of the previous embodiments wherein the aqueous solution has a pH of between 3.0 to 3.5 and the second co-solubilizing agent is selected from organic sugar, water soluble polymer, an acid, or an amino acid, or any combination thereof.

In embodiment 17, the present invention provides the cyclodextrin free pharmaceutical composition according to any one of the previous embodiments wherein the second co-solubilizing agent is dextrose, mannitol, glycine, an N-vinyl pyrrolidone polymer, butyric acid, adipic acid, phenylalanine, arginine HCl, tryptophan, or N-Acetyl tryptophan, or any combination thereof.

In embodiment 18, the present invention provides the cyclodextrin free pharmaceutical composition according to any one of the previous embodiments wherein the second co-solubilizing agent is an N-vinyl pyrrolidone polymer, mannitol, or glycine, or any combination thereof.

In embodiment 19, the present invention provides the cyclodextrin free pharmaceutical composition according to any one of the previous embodiments wherein the second co-solubilizing agent is 1-ethenylpyrrolidin-2-one (PVP; also called a polyvinylpyrrolidone), mannitol, or glycine, or any combination thereof. Various PVP are known to those skilled in the art, for example see https://www.brenntag.com/media/documents/bsi/product_data_sheets/material science/as hland_polymers/pvp_polymers_brochure.pdf. PVP is available in several grades of molecular weights and K-values (viscosity of 1% solution) such as PVP K-12, K-15, K17, K-30, K-60, K-90, or K-120.

In embodiment 20, the present invention provides the cyclodextrin free pharmaceutical composition according to any one of the previous embodiments wherein said PVP has a molecular weight ranges from 3,000 MW to 40,000 MW.

In embodiment 21, the present invention provides the cyclodextrin free pharmaceutical composition according to any one of the previous embodiments wherein said PVP has a molecular weight ranges from 10,000 MW to 17,000 MW.

In embodiment 22, the present invention provides the cyclodextrin free pharmaceutical composition according to any one of the previous embodiments wherein said PVP has a molecular weight ranges from 10,000 MW.

In embodiment 23, the present invention provides the cyclodextrin free pharmaceutical composition according to any one of the previous embodiments wherein said PVP is selected from the group consisting of 24% PVP 10,000 MW, 29% PVP 10,000 MW, 10% PVP 12,000 MW, 20% PVP 12,000 MW, 24% PVP 12,000 MW, or 24% PVP 17,000 MW.

In embodiment 24, the present invention provides the cyclodextrin free pharmaceutical composition according to any one of the previous embodiments wherein said composition comprises from 0.75% to 1% of dimethylsulfoxide, from 1.0% to 1.8% of PEG400, from 1.0% to 1.8% of ethanol, from 0.10% to 0.25% lactic acid, from 20% to 30% PVP 10,000 MW, and wherein the concentration of the carfilzomib is 2 mg/ml.

In embodiment 25, the present invention provides the cyclodextrin free pharmaceutical composition according to any one of the previous embodiments wherein said composition comprises from 0.2% to 2% of dimethylsulfoxide, from 0.5% to 2.5% of PEG400, from 0.5% to 2.5% of ethanol, from 0.05% to 0.5% lactic acid, from 10% to 40% PVP 10,000 MW, and wherein the concentration of the carfilzomib is 2 mg/ml.

In embodiment 26, the present invention provides the cyclodextrin free pharmaceutical composition according to any one of the previous embodiments wherein said composition comprises 0.7%, 0.75%, 0.8%, 0.85%, 0.9%, 0.95%, 1.0%, 1.05%, 1.1%, 1.15%, or 1.2% of dimethylsulfoxide; 1%, 1.1%, 1.2%, 1.3%, 1.4%, 1.5%, and 1.6% of PEG400; 1.1%, 1.2%, 1.3%, 1.4%, 1.5%, and 1.6% of ethanol; 0.1%, 0.15%, 0.2%, 0.25%, 0.3%, and 0.35% lactic acid; about 25%, 26%, 27%, 28%, 29%, 30%, 31%, 32%, and 33% PVP 10,000 MW; and wherein the concentration of the carfilzomib is 2 mg/ml.

In embodiment 27, the present invention provides the cyclodextrin free pharmaceutical composition according to any one of the previous embodiments wherein said composition comprises about 0.85% of dimethylsulfoxide, about 1.4% of PEG400, about 1.4% of ethanol, about 0.15% lactic acid, about 28.8% PVP 10,000 MW, and wherein the concentration of the carfilzomib is 2 mg/ml.

In embodiment 28, the present invention provides the cyclodextrin free pharmaceutical composition according to any one of the previous embodiments wherein said composition has a solution osmolality of from 200 mOsmo to 600 mOsmo.

In embodiment 29, the present invention provides the cyclodextrin free pharmaceutical composition according to any one of the previous embodiments wherein said composition has a solution osmolality of from 250 mOsmo to 400 mOsmo.

In embodiment 30, the present invention provides the cyclodextrin free pharmaceutical composition according to any one of the previous embodiments wherein said composition has a solution osmolality of from 280 mOsmo to 320 mOsmo.

In embodiment 31, the present invention provides the cyclodextrin free pharmaceutical composition according to any one of the previous embodiments wherein said composition has a solution osmolality of 280, 290, 300, 310, or 320 mOsmo.

In embodiment 32, the present invention provides the cyclodextrin free pharmaceutical composition according to any one of the previous embodiments wherein said composition is a ready-to-use injection.

In embodiment 33, the present invention provides the cyclodextrin free pharmaceutical composition according to any one of the previous embodiments wherein said composition is obtained as a lyophilized powder or cake.

In embodiment 34, the present invention provides the cyclodextrin free pharmaceutical composition according to embodiment 33, wherein said lyophilized powder or cake can be reconstituted in less than 5 minutes.

In embodiment 35, the present invention provides a carfilzomib injection kit comprising:
(i) a product vial pharmaceutical composition comprising a stable lyophilized powder or cake prepared by a process comprising the steps of:
   (a) dissolving said carfilzomib or a pharmaceutically acceptable salt thereof in dimethylsulfoxide, a mixture of C₁₋₄ alkyl alcohol and polyethylene glycol, and lactic acid; to form a solution wherein the concentration of the carfilzomib or said salt thereof ranges between 20 mg/ml to 50 mg/ml;
   (b) diluting said carfilzomib solution with an acidic aqueous solution of water-soluble polymer and sugar mixture having a pH of between 2.5 to 4.5 to form a solution wherein the concentration of the carfilzomib or said salt thereof ranges between 1 mg/ml to 3 mg/ml; and
   (c) freeze drying the solution obtained in step (b); and;
(ii) a reconstitution vial composition comprising sterilized water,
wherein said pharmaceutical composition is cyclodextrin free and the injection is administered intravenously or subcutaneously.

In embodiment 36, the present invention provides a carfilzomib injection kit according to embodiment 35 wherein said water-soluble polymer is 1-ethenylpyrrolidin-2-one (PVP) and said sugar is mannitol or glycine or combination thereof.

In embodiment 37, the present invention provides a carfilzomib injection kit according to embodiment 36 wherein said water-soluble polymer is PVP having a molecular weight ranges from 3,000 MW to 40,000 MW; 10,000 MW to 17,000 MW; or 10,000 MW; and said sugar is mannitol or glycine.

In embodiment 38, the present invention provides a carfilzomib injection kit according to embodiment 36 wherein said water-soluble polymer is 24% PVP 10,000 MW, 29% PVP 10,000 MW, 10% PVP 12,000 MW, 20% PVP 12,000 MW, 24% PVP 12,000 MW, or 24% PVP 17,000 MW, and said sugar is mannitol.

In embodiment 39, the present invention provides a carfilzomib injection kit according to embodiment 36 wherein said water-soluble polymer is 20% PVP 12,000 MW, or 24% PVP 12,000 MW, and said sugar is mannitol.

In embodiment 40, the present invention provides a carfilzomib injection kit according to embodiment 36 wherein said water-soluble polymer is 20% PVP 12,000 MW, and said sugar is mannitol.

In embodiment 41, the present invention provides a carfilzomib injection kit according to embodiment 36 wherein in step (b) the pH of said acidic aqueous solution ranges between 3.0-3.5.

In embodiment 42, the present invention provides a carfilzomib injection kit according to embodiment 36 wherein the solution formed in step (b) comprises from 0.75% to 1% of dimethylsulfoxide, from 1.0% to 1.8% of PEG400, from 1.0% to 1.8% of ethanol, from 0.10% to 0.25% lactic acid, from 20% to 30% PVP 10,000 MW, and wherein the concentration of the carfilzomib is 2 mg/ml.

In embodiment 43, the present invention provides a carfilzomib injection kit according to embodiment 36 wherein the solution formed in step (b) comprises from 0.2% to 2% of dimethylsulfoxide, from 0.5% to 2.5% of PEG400, from 0.5% to 2.5% of ethanol, from 0.05% to 0.5% lactic acid, from 10% to 40% PVP 10,000 MW, and wherein the concentration of the carfilzomib is 2 mg/ml.

In embodiment 44, the present invention provides a carfilzomib injection kit according to embodiment 36 wherein the solution formed in step (b) comprises 0.7%, 0.75%, 0.8%, 0.85%, 0.9%, 0.95%, 1.0%, 1.05%, 1.1%, 1.15%, or 1.2% of dimethylsulfoxide; 1%, 1.1%, 1.2%, 1.3%, 1.4%, 1.5%, and 1.6% of PEG400; 1.1%, 1.2%, 1.3%, 1.4%, 1.5%, and 1.6% of ethanol; 0.1%, 0.15%, 0.2%, 0.25%, 0.3%, and 0.35% lactic acid; about 25%, 26%, 27%, 28%, 29%, 30%, 31%, 32%, and 33% PVP 10,000 MW; and wherein the concentration of the carfilzomib is 2 mg/ml.

In embodiment 45, the present invention provides a carfilzomib injection kit according to embodiment 36 wherein the solution formed in step (b) comprises about 0.85% of dimethylsulfoxide, about 1.4% of PEG400, about 1.4% of ethanol, about 0.15% lactic acid, about 28.8% PVP 10,000 MW, and wherein the concentration of the carfilzomib is 2 mg/ml.

In embodiment 46, the present invention provides a carfilzomib injection kit according to embodiment 36 wherein the solution formed in step (b) has a solution osmolality of from 200 mOsmo to 600 mOsmo.

In embodiment 47, the present invention provides a carfilzomib injection kit according to embodiment 36 wherein the solution formed in step (b) has a solution osmolality of from 250 mOsmo to 400 mOsmo.

In embodiment 48, the present invention provides a carfilzomib injection kit according to embodiment 36 wherein the solution formed in step (b) has a solution osmolality of from 280 mOsmo to 320 mOsmo.

In embodiment 49, the present invention provides a carfilzomib injection kit according to embodiment 36 wherein the solution formed in step (b) has a solution osmolality of 280, 290, 300, 310, or 320 mOsmo.

In embodiment 50, the present invention provides a carfilzomib injection kit according to embodiment 36 wherein in step (b) the concentration of the carfilzomib or said salt thereof is 2 mg/ml.

In embodiment 51, the present invention provides a carfilzomib injection kit according to embodiment 36 wherein the ratio of lactic acid to the carfilzomib is 1.5:2 by weight.

In embodiment 52, the present invention provides a carfilzomib injection kit according to embodiment 36 wherein the ratio of lactic acid to the carfilzomib is 0.4:2 by weight.

In embodiment 53, the present invention provides a carfilzomib injection kit according to embodiment 36 wherein the injection is administered intravenously.

In embodiment 54, the present invention provides a carfilzomib injection kit according to embodiment 36 wherein the injection is administered subcutaneously.

In embodiment 55, the present invention provides a process for preparation of a cyclodextrin free carfilzomib lyophilized powder or cake suitable for injection upon reconstitution comprising the steps of:
(a) dissolving the carfilzomib or a pharmaceutically acceptable salt thereof in dimethylsulfoxide, a mixture of C₁₋₄ alkyl alcohol and polyethylene glycol, and lactic acid; to form a solution wherein the concentration of the carfilzomib or said salt thereof ranges between 20 mg/ml to 50 mg/ml;
(b) diluting the carfilzomib solution with an acidic aqueous solution of water-soluble polymer and sugar mixture having a pH of between 2.5 to 4.5 to form a solution wherein the concentration of the carfilzomib or said salt thereof ranges between 1 mg/ml to 3 mg/ml; and
(c) freeze drying the solution obtained in step (b).

In embodiment 56, the present invention provides the process according to claim 55, wherein said water-soluble polymer is 1-ethenylpyrrolidin-2-one (PVP) and said sugar is mannitol or glycine or combination thereof.

In embodiment 57, the present invention provides the process according to claim 55, wherein said water-soluble polymer is PVP having a molecular weight ranges from 3,000 MW to 40,000 MW; 10,000 MW to 17,000 MW; or 10,000 MW; and said sugar is mannitol or glycine.

In embodiment 58, the present invention provides the process according to claim 55, wherein said water-soluble polymer is 24% PVP 10,000 MW, 29% PVP 10,000 MW, 10% PVP 12,000 MW, 20% PVP 12,000 MW, 24% PVP 12,000 MW, or 24% PVP 17,000 MW, and said sugar is mannitol.

In embodiment 59, the present invention provides the process according to claim 55, wherein said water-soluble polymer is 20% PVP 12,000 MW, or 24% PVP 12,000 MW, and said sugar is mannitol.

In embodiment 60, the present invention provides the process according to

claim 55, wherein said water-soluble polymer is 20% PVP 12,000 MW, and said sugar is mannitol.

In embodiment 61, the present invention provides the process according to claim 55, wherein in step (b) the pH of said acidic aqueous solution ranges between 3.0-3.5.

In embodiment 62, the present invention provides the process according to claim 55, wherein the solution formed in step (b) comprises from 0.75% to 1% of dimethylsulfoxide, from 1.0% to 1.8% of PEG400, from 1.0% to 1.8% of ethanol, from 0.10% to 0.25% lactic acid, from 20% to 30% PVP 10,000 MW, and wherein the concentration of the carfilzomib is 2 mg/ml.

In embodiment 63, the present invention provides the process according to claim 55, wherein the solution formed in step (b) comprises from 0.2% to 2% of dimethylsulfoxide, from 0.5% to 2.5% of PEG400, from 0.5% to 2.5% of ethanol, from 0.05% to 0.5% lactic acid, from 10% to 40% PVP 10,000 MW, and wherein the concentration of the carfilzomib is 2 mg/ml.

In embodiment 64, the present invention provides the process according to claim 55, wherein the solution formed in step (b) comprises 0.7%, 0.75%, 0.8%, 0.85%, 0.9%, 0.95%, 1.0%, 1.05%, 1.1%, 1.15%, or 1.2% of dimethylsulfoxide; 1%, 1.1%, 1.2%, 1.3%, 1.4%, 1.5%, and 1.6% of PEG400; 1.1%, 1.2%, 1.3%, 1.4%, 1.5%, and 1.6% of ethanol; 0.1%, 0.15%, 0.2%, 0.25%, 0.3%, and 0.35% lactic acid; about 25%, 26%, 27%, 28%, 29%, 30%, 31%, 32%, and 33% PVP 10,000 MW; and wherein the concentration of the carfilzomib is 2 mg/ml.

In embodiment 65, the present invention provides the process according to claim 55, wherein the solution formed in step (b) comprises about 0.85% of dimethylsulfoxide, about 1.4% of PEG400, about 1.4% of ethanol, about 0.15% lactic acid, about 28.8% PVP 10,000 MW, and wherein the concentration of the carfilzomib is 2 mg/ml.

In embodiment 66, the present invention provides the process according to claim 55, wherein the solution formed in step (b) has a solution osmolality of from 200 mOsmo to 600 mOsmo.

In embodiment 67, the present invention provides the process according to claim 55, wherein the solution formed in step (b) has a solution osmolality of from 250 mOsmo to 400 mOsmo.

In embodiment 68, the present invention provides the process according to

claim 55, wherein the solution formed in step (b) has a solution osmolality of from 280 mOsmo to 320 mOsmo.

In embodiment 69, the present invention provides the process according to claim 55, wherein the solution formed in step (b) has a solution osmolality of 280, 290, 300, 310, or 320 mOsmo.

In embodiment 70, the present invention provides the process according to claim 55, wherein in step (b) the concentration of the carfilzomib or said salt thereof is 2 mg/ml.

In embodiment 71, the present invention provides the process according to claim 55, wherein in step (b) the ratio of lactic acid to the carfilzomib is 1.5:2 by weight.

In embodiment 72, the present invention provides the process according to claim 55, wherein in step (b) the ratio of lactic acid to the carfilzomib is 0.4:2 by weight.

In embodiment 73, the present invention provides the process according to claim 55, wherein the injection is administered intravenously.

In embodiment 74, the present invention provides the process according to claim 55, wherein the injection is administered subcutaneously.

In embodiment 75, the present invention provides a method of treating multiple myeloma in a subject in need of treatment, comprising administering a therapeutically effective amount of the cyclodextrin free pharmaceutical composition of any one of embodiments 1 to 34, or the kit of any one of embodiments 35 to 54.

In embodiment 76, the present invention provides the method according to embodiment 75 further comprising simultaneous, sequential, or separate administration of a therapeutically effective amount of a chemotherapeutic agent.

In embodiment 77, the present invention provides a method of treating a solid tumor in a subject in need of treatment, comprising administering a therapeutically effective amount of the cyclodextrin free pharmaceutical composition of any one of embodiments 1 to 34, or the kit of any one of embodiments 35 to 54.

In embodiment 78, the present invention provides the method according to embodiment 77, further comprising simultaneous, sequential, or separate administration of a therapeutically effective amount of a chemotherapeutic agent.

In embodiment 79, the present invention provides a carfilzomib injection kit comprising: (a) a stable frozen carfilzomib or a pharmaceutically acceptable salt thereof pharmaceutical composition and (b) a dissolution pharmaceutical composition, wherein said kit is prepared by a process comprising the steps of:
(i) dissolving said carfilzomib or a pharmaceutically acceptable salt thereof in dimethylsulfoxide (DMSO) to form a DMSO solution wherein the concentration of the carfilzomib or said salt thereof ranges between 200 mg/ml to 250 mg/ml;
(ii) freezing said DMSO solution at 2° C. to 8° C. to form said frozen carfilzomib pharmaceutical composition; and optionally storing the frozen composition at 2° C. to 8° C.;
(iii) thawing said frozen carfilzomib pharmaceutical composition at DMSO melting point, preferable at least at a temperature of 18° C., to form a thawed carfilzomib composition, and mixing said liquid carfilzomib composition with the dissolution pharmaceutical composition to form a solution wherein the concentration of the carfilzomib ranges between 1 mg/ml to 3 mg/ml;
   wherein said pharmaceutical composition is cyclodextrin free and the injection is administered intravenously or subcutaneously.

In embodiment 80, the present invention provides the kit of embodiment 79 wherein said dissolution pharmaceutical composition includes a co-solvent vial which can dissolve said thawed carfilzomib pharmaceutical composition to form a solution wherein the concentration of the carfilzomib ranges between 20 mg/ml to 50 mg/ml; and an additional excipient vial which can dissolve said thawed carfilzomib pharmaceutical composition to form a solution wherein the concentration of the carfilzomib ranges between 1 mg/ml to 3 mg/ml.

In embodiment 81, the present invention provides the kit of embodiment 80 wherein the frozen carfilzomib composition is stored at 2° C. to 8° C.; and said diluting step (iii) is performed in the clinic facility.

In embodiment 82, the present invention provides the kit of embodiment 81 wherein the frozen carfilzomib composition is stored in a moisture-free storage container or a device.

In embodiment 83, the present invention provides the kit of embodiment 82 wherein said moisture-free container is a 0.5 mL microcentrifuge Eppendorf tube and 3 cc glass Schott 1A vial with lyophilized stoppers and crimp seals.

In embodiment 84, the present invention provides the kit of embodiment 80 wherein said co-solvent vial contains a co-solvent system selected from C₁₋₄ alkyl alcohol, polyethylene glycol, or combination thereof optionally in the present of a first co-solubilizing agent.

In embodiment 85, the present invention provides the kit of embodiment 80 wherein said co-solvent vial contains a co-solvent system selected from ethanol and PEG combination in the presence of a first co-solubilizing agent, which is lactic acid.

In embodiment 86, the present invention provides the kit of embodiment 80 wherein said additional excipient vial contains an aqueous solution having a pH of between 2.5 to 4.5 optionally in the present of a second co-solubilizing agent, which is a water-soluble polymer.

In embodiment 87, the present invention provides the kit of embodiment 86 wherein said water-soluble polymer is 1-ethenylpyrrolidin-2-one (PVP).

In embodiment 88, the present invention provides the kit of embodiment 87 wherein said water-soluble polymer is PVP having a molecular weight ranges from 3,000 MW to 40,000 MW; 10,000 MW to 17,000 MW; or 10,000 MW.

In embodiment 89, the present invention provides the kit of embodiment 87 wherein said water-soluble polymer is 24% PVP 10,000 MW, 29% PVP 10,000 MW, 10% PVP 12,000 MW, 20% PVP 12,000 MW, 24% PVP 12,000 MW, or 24% PVP 17,000 MW.

In embodiment 90, the present invention provides the kit of embodiment 87 wherein said water-soluble polymer is 20% PVP 12,000 MW or 24% PVP 12,000 MW.

In embodiment 91, the present invention provides the kit of embodiment 87 wherein said water-soluble polymer is 20% PVP 12,000 MW.

In embodiment 92, the present invention provides the kit of embodiment 86

wherein the pH of said aqueous solution ranges between 3.0-3.5.

In embodiment 93, the present invention provides the kit of embodiment 87 wherein the solution formed in step (iii) comprises from 0.75% to 1% of dimethylsulfoxide, from 1.0% to 1.8% of PEG400, from 1.0% to 1.8% of ethanol, from 0.10% to 0.25% lactic acid, from 20% to 30% PVP 10,000 MW, and wherein the concentration of the carfilzomib is 2 mg/ml.

In embodiment 94, the present invention provides the kit of embodiment 87 wherein the solution formed in step (iii) comprises from 0.2% to 2% of dimethylsulfoxide, from 0.5% to 2.5% of PEG400, from 0.5% to 2.5% of ethanol, from 0.05% to 0.5% lactic acid, from 10% to 40% PVP 10,000 MW, and wherein the concentration of the carfilzomib is 2 mg/ml.

In embodiment 95, the present invention provides the kit of embodiment 87 wherein the solution formed in step (iii) comprises 0.7%, 0.75%, 0.8%, 0.85%, 0.9%, 0.95%, 1.0%, 1.05%, 1.1%, 1.15%, or 1.2% of dimethylsulfoxide; 1%, 1.1%, 1.2%, 1.3%, 1.4%, 1.5%, and 1.6% of PEG400; 1.1%, 1.2%, 1.3%, 1.4%, 1.5%, and 1.6% of ethanol; 0.1%, 0.15%, 0.2%, 0.25%, 0.3%, and 0.35% lactic acid; about 25%, 26%, 27%, 28%, 29%, 30%, 31%, 32%, and 33% PVP 10,000 MW; and wherein the concentration of the carfilzomib is 2 mg/ml.

In embodiment 96, the present invention provides the kit of embodiment 87 wherein the solution formed in step (iii) comprises about 0.85% of dimethylsulfoxide, about 1.4% of PEG400, about 1.4% of ethanol, about 0.15% lactic acid, about 28.8% PVP 10,000 MW, and wherein the concentration of the carfilzomib is 2 mg/ml.

In embodiment 97, the present invention provides the kit of embodiment 87 wherein the solution formed in step (iii) has a solution osmolality of from 200 mOsmo to 600 mOsmo.

In embodiment 98, the present invention provides the kit of embodiment 87 wherein the solution formed in step (iii) has a solution osmolality of from 250 mOsmo to 400 mOsmo.

In embodiment 99, the present invention provides the kit of embodiment 87 wherein the solution formed in step (iii) has a solution osmolality of from 280 mOsmo to 320 mOsmo.

In embodiment 100, the present invention provides the kit of embodiment 87 wherein the solution formed in step (iii) has a solution osmolality of 280, 290, 300, 310, or 320 mOsmo.

In embodiment 101, the present invention provides the kit of embodiment 87 wherein the solution formed in step (iii), the concentration of the carfilzomib or said salt thereof is 2 mg/ml.

In embodiment 102, the present invention provides the kit of embodiment 87 wherein the ratio of lactic acid to the carfilzomib is 1.5:2 by weight.

In embodiment 103, the present invention provides the kit of embodiment 87 wherein the ratio of lactic acid to the carfilzomib is 0.4:2 by weight.

In embodiment 104, the present invention provides the kit of embodiment 87 wherein the injection is administered intravenously.

In embodiment 105, the present invention provides the kit of embodiment 87 wherein the injection is administered subcutaneously.

In embodiment 106, the present invention provides a process for preparation of a cyclodextrin free frozen carfilzomib composition comprising the step of:
(i) dissolving said carfilzomib or a pharmaceutically acceptable salt thereof in dimethylsulfoxide (DMSO) to form a DMSO solution wherein the concentration of the carfilzomib or said salt thereof ranges between 200 mg/ml to 250 mg/ml;
(ii) freezing said DMSO solution at 2° C. to 8° C. to form said frozen carfilzomib pharmaceutical composition; and optionally storing the frozen composition at 2° C. to 8° C.

In embodiment 107, the present invention provides the process of embodiment 106 further comprising the steps of: thawing said frozen carfilzomib pharmaceutical composition at DMSO melting point, preferable at least at a temperature of 18° C., to form a thawed carfilzomib composition, and mixing said liquid carfilzomib composition with a dissolution pharmaceutical composition to form a solution wherein the concentration of the carfilzomib ranges between 1 mg/ml to 3 mg/ml; wherein said pharmaceutical composition is cyclodextrin free and is suitable for injection.

In embodiment 108, the present invention provides the process of embodiment 107 wherein said dissolution pharmaceutical composition comprises a mixture of C₁₋₄ alkyl alcohol and polyethylene glycol in the presence of lactic acid; to form a solution wherein the concentration of the carfilzomib or said salt thereof ranges between 20 mg/ml to 50 mg/ml.

In embodiment 109, the present invention provides the process of embodiment 107 wherein said dissolution pharmaceutical composition further comprises a second vial comprising an acidic aqueous solution of water-soluble polymer having a pH of between 2.5 to 4.5 which can further dilute said solution to form a more diluted solution wherein the concentration of the carfilzomib or said salt thereof ranges between 1 mg/ml to 3 mg/ml.

In embodiment 110, the present invention provides the process of embodiment 107 wherein said water-soluble polymer is 1-ethenylpyrrolidin-2-one (PVP).

In embodiment 111, the present invention provides the process of embodiment 107 wherein said water-soluble polymer is PVP having a molecular weight ranges from 3,000 MW to 40,000 MW; 10,000 MW to 17,000 MW; or 10,000 MW.

In embodiment 112, the present invention provides the process of embodiment

107 wherein said water-soluble polymer is 24% PVP 10,000 MW, 29% PVP 10,000 MW, 10% PVP 12,000 MW, 20% PVP 12,000 MW, 24% PVP 12,000 MW, or 24% PVP 17,000 MW.

In embodiment 113, the present invention provides the process of embodiment 107 wherein said water-soluble polymer is 20% PVP 12,000 MW, or 24% PVP 12,000 MW.

In embodiment 114, the present invention provides the process of embodiment 109 wherein said water-soluble polymer is 20% PVP 12,000 MW.

In embodiment 115, the present invention provides the process of embodiment 109 wherein the pH of said acidic aqueous solution ranges between 3.0-3.5.

In embodiment 116, the present invention provides the process of embodiment 109 wherein the more diluted solution comprises from 0.75% to 1% of dimethylsulfoxide, from 1.0% to 1.8% of PEG400, from 1.0% to 1.8% of ethanol, from 0.10% to 0.25% lactic acid, from 20% to 30% PVP 10,000 MW, and wherein the concentration of the carfilzomib is 2 mg/ml.

In embodiment 117, the present invention provides the process of embodiment 109 wherein the more diluted solution comprises from 0.2% to 2% of dimethylsulfoxide, from 0.5% to 2.5% of PEG400, from 0.5% to 2.5% of ethanol, from 0.05% to 0.5% lactic acid, from 10% to 40% PVP 10,000 MW, and wherein the concentration of the carfilzomib is 2 mg/ml.

In embodiment 118, the present invention provides the process of embodiment 109 wherein the more diluted solution comprises 0.7%, 0.75%, 0.8%, 0.85%, 0.9%, 0.95%, 1.0%, 1.05%, 1.1%, 1.15%, or 1.2% of dimethylsulfoxide; 1%, 1.1%, 1.2%, 1.3%, 1.4%, 1.5%, and 1.6% of PEG400; 1.1%, 1.2%, 1.3%, 1.4%, 1.5%, and 1.6% of ethanol; 0.1%, 0.15%, 0.2%, 0.25%, 0.3%, and 0.35% lactic acid; about 25%, 26%, 27%, 28%, 29%, 30%, 31%, 32%, and 33% PVP 10,000 MW; and wherein the concentration of the carfilzomib is 2 mg/ml.

In embodiment 119, the present invention provides the process of embodiment 109 wherein the more diluted solution comprises about 0.85% of dimethylsulfoxide, about 1.4% of PEG400, about 1.4% of ethanol, about 0.15% lactic acid, about 28.8%

PVP 10,000 MW, and wherein the concentration of the carfilzomib is 2 mg/ml.

In embodiment 120, the present invention provides the process of embodiment 109 wherein the more diluted solution has a solution osmolality of from 200 mOsmo to 600 mOsmo.

In embodiment 121, the present invention provides the process of embodiment

109 wherein the more diluted solution has a solution osmolality of from 250 mOsmo to 400 mOsmo.

In embodiment 122, the present invention provides the process of embodiment 109 wherein the more diluted solution has a solution osmolality of from 280 mOsmo to 320 mOsmo.

In embodiment 123, the present invention provides the process of embodiment 109 wherein the more diluted solution has a solution osmolality of 280, 290, 300, 310, or 320 mOsmo.

In embodiment 124, the present invention provides the process of embodiment 109 wherein in said more diluted solution the concentration of the carfilzomib or said salt thereof is 2 mg/ml.

In embodiment 125, the present invention provides the process of embodiment 109 wherein in said more diluted solution the ratio of lactic acid to the carfilzomib is 1.5:2 by weight.

In embodiment 126, the present invention provides the process of embodiment 109 wherein in said more diluted solution the ratio of lactic acid to the carfilzomib is 0.4:2 by weight.

In embodiment 127, the present invention provides the process of embodiment 109 wherein the injection is administered intravenously.

In embodiment 128, the present invention provides the process of embodiment 109 wherein the injection is administered subcutaneously.

In embodiment 129, the present invention provides a method of treating multiple myeloma in a subject in need of treatment, comprising administering a therapeutically effective amount of the carfilzomib solution obtained from said injection kit of any one of embodiments 79-105.

In embodiment 130, the present invention provides the method of embodiment 129, further comprising simultaneous, sequential, or separate administration of a therapeutically effective amount of a chemotherapeutic agent.

In embodiment 131, the present invention provides a method of treating a solid tumor in a subject in need of treatment, comprising administering a therapeutically effective amount of the carfilzomib solution obtained from said injection kit of any one of embodiments 79-105.

In embodiment 132, the present invention provides the method of embodiment 131, further comprising simultaneous, sequential, or separate administration of a therapeutically effective amount of a chemotherapeutic agent.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

FIG. 1 illustrates a visual comparison of carfilzomib active ingredient in (a) water, (b) cyclodextrin (CAPTISOL®) and cyclodextrin-free formulation of the present invention

FIG. 2 illustrates 3D solubility plot of the solvent sphere for solubilizing CFZ-API.

FIG. 3 illustrates 2D solubility plots comparing each solubility parameter for solubilizing CFZ-API.

FIG. 4 illustrates a list of solvents generated by Hansen Solubility Parameter

software having δD=16 to 19.5; δP=5 to 18; and δH=7 to 19.6.

FIG. 5 illustrates a list of solvents generated by Hansen Solubility Parameter software having δD=16 to 24; δP=8 to 14; and δH=17 to 24.

FIG. 6 illustrates no significant percent main peak loss measured by RP-HPLC of liquid, CAPTISOL® free formulations at a temperature of 2° C. to 8° C. (A) and 25° C. (B).

FIG. 7 illustrates visual differences between frozen carfilzomib drug product in containers with and without crimp seals at 4 weeks storage at 2° C. to 8° C.

FIG. 8 illustrates frozen CFZ-API at 2-8° C. High concentration CFZ-API in DMSO had no significant loss of percent main peak at 4 weeks, as measured by RP-HPLC.

FIG. 9 illustrates proteasomal activity of subcutaneously administered CAPTISOL® free carfilzomib formulation in mice.

FIG. 10 illustrates proteasomal activity of intravenously administered CAPTISOL® free carfilzomib formulation in mice.

### DETAILED DESCRIPTION

### Definitions

The term “C_{x-y}alkyl” refers to unsubstituted saturated hydrocarbon groups, including straight-chain alkyl and branched-chain alkyl groups that contain from x to y carbons in the chain.

The terms “amine” and “amino” are art-recognized and refer to both unsubstituted and substituted amines and salts thereof, e.g., a moiety that can be represented by the general formulae:

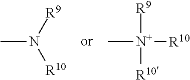

where R⁹, R¹⁰ and R^{10′} each independently represent a hydrogen, an alkyl, an alkenyl, —(CH₂)ₘ—R⁸, or R⁹ and R¹⁰ taken together with the N atom to which they are attached complete a heterocycle having from 4 to 8 atoms in the ring structure; R⁸ represents an aryl, a cycloalkyl, a cycloalkenyl, a heterocyclyl or a polycyclyl; and m is zero or an integer from 1 to 8. In some embodiments, only one of R⁹ or R¹⁰ is a carbonyl, e.g., R⁹, R¹⁰, and the nitrogen together do not form an imide. In some embodiments, R⁹ and R¹⁰ (and optionally R^{10′}) each independently represent a hydrogen, an alkyl, an alkenyl, or —(CH₂)ₘ—R⁸. In certain embodiments, an amino group is basic, meaning its protonated form has a pKa above 7.00.

The term “buffer” is a substance which by its presence in solution increases the amount of acid or alkali that must be added to cause a unit change in pH. Thus, a buffer is a substance that assists in regulating the pH of a composition. Typically, a buffer is chosen based upon the desired pH and compatibility with other components of a composition. In general, a buffer has a pKa that is no more than 1 unit less than or greater than the desired pH of the composition (or that the composition will produce upon dissolution).

The term “water” as used herein refers to a liquid solution of H₂O having a pH of approximately 7.0.

The term “C_{x-y}alkyl alcohol” refers to a C_{x-y}alkyl group substituted with a hydroxy group.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents, and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As used herein, the term “peptide” refers to a chain of amino acids that is about two to about ten amino acids in length.

As used herein, the term “natural” or “naturally occurring” amino acid refers to one of the twenty most common occurring amino acids. Natural amino acids are referred to by their standard one- or three-letter abbreviations.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “proteasome” as used herein is meant to include immuno- and constitutive proteasomes.

As used herein, the term “inhibitor” is meant to describe a compound that blocks or reduces an activity of an enzyme or system of enzymes, receptors, or other pharmacological target (for example, inhibition of proteolytic cleavage of standard fluorogenic peptide substrates such as suc-LLVY-AMC, Box-LLR-AMC and Z-LLE-AMC, inhibition of various catalytic activities of the 20S proteasome). An inhibitor can act with competitive, uncompetitive, or noncompetitive inhibition. An inhibitor can bind reversibly or irreversibly, and therefore the term includes compounds that are suicide substrates of an enzyme. An inhibitor can modify one or more sites on or near the active site of the enzyme, or it can cause a conformational change elsewhere on the enzyme. The term inhibitor is used more broadly herein than scientific literature so as to also encompass other classes of pharmacologically or therapeutically useful agents, such as agonists, antagonists, stimulants, co-factors, and the like.

As used herein, “low solubility” refers to being sparingly soluble, slightly soluble, very slightly soluble, practically insoluble, or insoluble in, for example, water or another solution (e.g., a first combination); the terms “sparingly soluble, slightly soluble, very slightly soluble, practically insoluble, or insoluble” correspond in meaning to the United States Pharmacopeia (USP) general terms for approximate solubility expression. See, e.g., DeLuca and Boylan in Pharmaceutical Dosage Forms: Parenteral Medications, vol. 1, Avis, K. E., Lackman, L. and Lieberman, H. A., eds; Marcel Dekkar: 1084, pages 141-142:

| | Relative amount of solvent |
|---|---|
| USP term | to dissolve 1 part of solute |
| Sparingly soluble | 30-100 |
| Slightly soluble | 100-1,000 |
| Very slightly soluble | 1,000-10,000 |
| Practically insoluble, or insoluble | >10,000 |

“Heterogeneous” as used herein refers to a solution having a non-uniform (multiphase) composition. For example, a heterogeneous solution can include a suspension of solid particles in a liquid (e.g., a slurry).

“Homogeneous” as used herein refers to a solution that is consistent or uniform throughout its volume (single phase, observed as clear solution).

A “therapeutically effective amount” of a compound with respect to the subject method of treatment, refers to an amount of the compound(s) in a preparation which, when administered as part of a desired dosage regimen (to a patient, e.g., a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in manner to improve or stabilize a patient's condition.

Many small molecule organic compound drugs have pH dependent solubility. It is frequent that a pH range appropriate for administration of a drug (such as by injection where the tolerable pH range is generally considered from pH 3 to pH 10.5 for intravenous administration) is not in the same pH where sufficient solubility of the drug can be found in aqueous solution (for example at or below pH 2). To enable a pharmaceutically useful concentration level of drug in solution at a pH range acceptable and tolerable for administration (e.g. by injection), the order of the solvent addition and pH adjustment as aqueous solution is introduced are useful considerations to the present formulation as claimed here.

For basic drug molecules, solubility is usually enhanced at lower pH, which also presents stability and shelf life challenges in some instances if used without cyclodextrin(s). For example, sufficient solubility may be achieved via lowering the pH of a solution with an acid, however such pH reduction may lead to degradation reactions from the acidic conditions. See Table 1 for intrinsic aqueous solubility data for carfilzomib, showing some moderate increase in solubility with lowering of pH.

TABLE 1
Aqueous solubility of CFZ-API as a function
of pH, without cyclodextrins

| Solvent | Solubility (mg/ml) |
|---|---|
| Water/pH 7 | 0.002 |
| Water/pH 5 | 0.002 |
| Water/pH 3 | 0.02 |
| Water/pH 1 | 1.8 |

Numerous acid mediated degradation reaction pathways exist for small molecule drugs and biological molecules, such as hydrolysis of amides in smaller inactive peptide fragments, or hydrolytic opening of functional epoxides moieties. The products of acid mediated degradation may lack pharmacological activity and may be toxic or genotoxic compounds even at trace levels. It is therefore helpful that the CFZ-API is entirely dissolved in the solvent, such as N-methyl-2-pyrrolidone (NMP) or dimethyl sulfoxide (DMSO), and co-solvent mixture of the invention prior to introduction of the aqueous solution with appropriate pH.

In order to balance the competing needs of avoiding acid mediated degradation side reactions which occur at low pH, the present inventors found a unique pH condition coupled with addition of a soluble polymer co-solubilizing agent. Surprisingly, the pH of an aqueous solution achieved via the addition of certain concentrations of acids, for example methane sulfonic acid (around pH 3.0 to 3.5), in the presence of a soluble polymer co-solubilizing agent, preferably polymer which contains pyrrolidone ring or similar structure, such as polyvinylpyrrolidone (PVP) ring, preferably 10,000 MW PVP, which solubilizes 90% or more CFZ-API. Other co-solubilizing agents containing a pyrrolidone ring may work to solubilize and stabilize CFZ. Some examples of pyrrolidone agents that would be useful in solubilizing CFZ include methyl pyrrolidone (e.g., n-vinyl 3-methyl 2-pyrrolidone, n-vinyl 4-methyl 2-pyrrolidone, and n-vinyl 5-methyl pyrrolidone) as a solvent, monomer, or polymer.

Minimizing number of steps to dissolve CFZ-API into solution helps with ease of manufacturing as well as clinic handling. Different combination of water miscible solvents, co-solvents, acids, and water-containing solutions were mixed to simplify the multistep procedure. From the formulation test samples Nos. 1 to 3 discussed above, combinations of water miscible solvents and co-solvents were able to result in similar solubility of CFZ-API at ≥20 mg/ml. It was found from further screening that the introduction of the aqueous solvent step to further dilute the CFZ-API to ≥2 mg/ml could not be combined with water miscible solvents and/or co-solvents step because the CFZ-API would not dissolve. It was found that in order to maximize the solubility of the CFZ-API, each of the solvent mixture must be added in multi-steps pattern prior to the final option of the drug product presentation, which can be ready to use drug product or a lyophilized drug product. The flow scheme for multi-step preparation of the cyclodextrin free lyophilized drug product is described below:

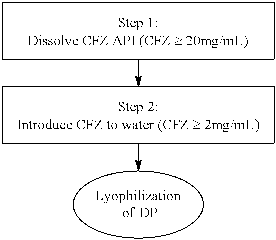

In Step 1 of the flow scheme preparation, non-aqueous solvents consisting of water miscible organic solvents and a co-solvent system were first added to dissolve the CFZ-API solid due its very low water solubility. In Step 2 of the flow scheme preparation, the non-aqueous CFZ-API solution is then introduced into an acidic, aqueous environment with a final pH 3.0 to 3.5 to obtain maximum CFZ-API solubility. The solution was then filtered by using 0.22 μm PES syringe filter equipped on a NORMJECT® (silicone free) syringe. The resulting filtrate was then checked for CFZ-API solubility recovery and stability on reverse phase high performance liquid chromatography (RP-HPLC). RP-HPLC was used to determine peak degradation as well as CFZ-API recovery through the use of a 3 to 5 point reference standard curve. Peak integration of standards was taken against standard buffer, 50% acetonitrile in water; while peak integration of formulation samples was taken against buffer of the formulation. In Step 3 of the diagram, lyophilization step was performed on the filtrate. Reconstitution of lyophilized product with water for injection (WFI) yielded CFZ-API solubility of about 1.5 mg/ml to 5 mg/ml in the preferred formulations of the invention.

A multi-step solvent addition which include a separate step of acidifying solution to get CFZ-API target concentration and pH was used to maximize CFZ-API solubility. Cyclodextrin-free CFZ-API formulations can be made in two step or three step schemes as shown below:

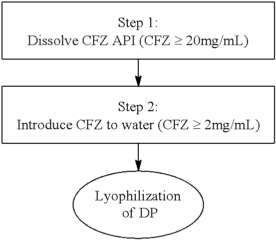

The first step can consist of dissolving the CFZ-API API into organic mixture to about 20 to 50 mg/ml. This organic mixture could consist of DMSO, PEG400, ethanol, and lactic acid. The second step can be to add an acidic, solubilizing-agent-containing solution included with water to final CFZ-API concentration of 2 mg/ml. This mixture could consist of povidone, water and MSA to pH about 2.9. The addition of this mixture to the first step containing CFZ-API should result in a partially precipitated to clear solution at about pH 3. An additional step to reach target pH of 3 with minimal MSA or MEA may be needed if step 2 did not reach target pH. Once the pH is achieved, filtration will be needed to filter excess CFZ-API that was not solubilized. Studies from these experiments suggest that the order of introducing these excipients to CFZ-API is important to obtain maximum CFZ-API solubility. For example, the aqueous mixture including povidone cannot be added to CFZ-API prior to the organic mixture because it will not dissolve the CFZ-API API.

Aside from lyophilization and injectable emulsions, another embodiment for cyclodextrin-free CFZ drug product presentation is in frozen form at 2° C. to 8° C. This option is advantageous because no lyophilizer or freezer would be needed to maintain high solubility and stability of CFZ. The composition can be made by dissolving CFZ API with DMSO to ≥200 mg/ml and stored at 2° C. to 8° C. to get a frozen product. This frozen state at higher temperature is due to the high melting temperature of DMSO, which is 19° C. The flow scheme for preparation of the frozen cyclodextrin free CFZ-API drug product pre- and post-manufacturing is described below. The first step would be performed by manufacturing. Storage and shipment at 2° C. to 8° C. would maintain the frozen state of the drug product. The clinic would receive steps 2 and 3 formulation solutions, or combinations thereof, to add into the thawed drug product upon clinical administration.

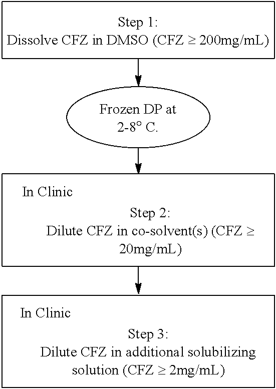

### Methods of Use

The biological applications of proteasome inhibition are numerous. Proteasome inhibition has been suggested as a prevention and/or treatment of a multitude of diseases including, but not limited to, proliferative diseases, neurotoxic/degenerative diseases, Alzheimer's, ischemic conditions, inflammation, auto-immune diseases, HIV, cancers, organ graft rejection, septic shock, inhibition of antigen presentation, decreasing viral gene expression, parasitic infections, conditions associated with acidosis, macular degeneration, pulmonary conditions, muscle wasting diseases, fibrotic diseases, bone and hair growth diseases. Therefore, pharmaceutical formulations for very potent, proteasome-specific compounds, such as the epoxy ketone class of molecules, provide a means of administering a drug to a patient and treating these conditions.

At the cellular level, the accumulation of polyubiquitinated proteins, cell morphological changes, and apoptosis have been reported upon treatment of cells with various proteasome inhibitors. Proteasome inhibition has also been suggested as a possible antitumor therapeutic strategy. The fact that epoxomicin was initially identified in a screen for antitumor compounds validates the proteasome as an antitumor chemotherapeutic target. Accordingly, these compositions are useful for treating cancer.

Both in vitro and in vivo models have shown that malignant cells, in general, are susceptible to proteasome inhibition. In fact, proteasome inhibition has already been validated as a therapeutic strategy for the treatment of multiple myeloma. This could be due, in part, to the highly proliferative malignant cell's dependency on the proteasome system to rapidly remove proteins (Rolfe et al., J. Mol. Med. (1997) 75:5-17; Adams, Nature (2004) 4:349-360). Therefore, provided herein is a method of treating cancers comprising administering to a patient in need of such treatment a therapeutically effective amount of a peptide proteasome inhibitor as provided herein.

As used herein, the term “cancer” includes, but is not limited to, blood born and solid tumors. Cancer refers to disease of blood, bone, organs, skin tissue and the vascular system, including, but not limited to, cancers of the bladder, blood, bone, brain, breast, cervix, chest, colon, endrometrium, esophagus, eye, head, kidney, liver, lung, lymph nodes, mouth, neck, ovaries, pancreas, prostate, rectum, renal, skin, stomach, testis, throat, and uterus. Specific cancers include, but are not limited to, leukemia (acute lymphocytic leukemia (ALL), acute lyelogenous leukemia (AML), chronic lymphocytic leukemia (CLL), chronic myelogenous leukemia (CML), hairy cell leukemia), mature B cell neoplasms (small lymphocytic lymphoma, B cell prolymphocytic leukemia, lymphoplasmacytic lymphoma (such as Waldenström's macroglobulinemia), splenic marginal zone lymphoma, plasma cell myeloma, plasmacytoma, monoclonal immunoglobulin deposition diseases, heavy chain diseases, extranodal marginal zone B cell lymphoma (MALT lymphoma), nodal marginal zone B cell lymphoma (NMZL), follicular lymphoma, mantle cell lymphoma, diffuse B cell lymphoma, mediastinal (thymic) large B cell lymphoma, intravascular large B cell lymphoma, primary effusion lymphoma and Burkitt lymphoma/leukemia), mature T cell and natural killer (NK) cell neoplasms (T cell prolymphocytic leukemia, T cell large granular lymphocytic leukemia, aggressive NK cell leukemia, adult T cell leukemia/lymphoma, extranodal NK/T cell lymphoma, enteropathy-type T cell lymphoma, hepatosplenic T cell lymphoma, blastic NK cell lymphoma, mycosis fungoides (Sezary syndrome), primary cutaneous anaplastic large cell lymphoma, lymphomatoid papulosis, angioimmunoblastic T cell lymphoma, unspecified peripheral T cell lymphoma and anaplastic large cell lymphoma), Hodgkin lymphoma (nodular sclerosis, mixed cellularity, lymphocyte-rich, lymphocyte depleted or not depleted, nodular lymphocyte-predominant), myeloma (multiple myeloma, indolent myeloma, smoldering myeloma), chronic myeloproliferative disease, myelodysplastic/myeloproliferative disease, myelodysplastic syndromes, immunodeficiency-associated lymphoproliferative disorders, histiocytic and dendritic cell neoplasms, mastocytosis, chondrosarcoma, Ewing sarcoma, fibrosarcoma, malignant giant cell tumor, myeloma bone disease, osteosarcoma, breast cancer (hormone dependent, hormone independent), gynecological cancers (cervical, endometrial, fallopian tube, gestational trophoblastic disease, ovarian, peritoneal, uterine, vaginal and vulvar), basal cell carcinoma (BCC), squamous cell carcinoma (SCC), malignant melanoma, dermatofibrosarcoma protuberans, Merkel cell carcinoma, Kaposi's sarcoma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, malignant mesothelioma (peritoneal mesothelioma, pericardial mesothelioma, pleural mesothelioma), gastro-entero-pancreatic or gastroenteropancreatic neuroendocrine tumor (GEP-NET), carcinoid, pancreatic endocrine tumor (PET), colorectal adenocarcinoma, colorectal carcinoma, aggressive neuroendocrine tumor, leiomyosarcoma mucinous adenocarcinoma, Signet Ring cell adenocarcinoma, hepatocellular carcinoma, cholangiocarcinoma, hepatoblastoma, hemangioma, hepatic adenoma, focal nodular hyperplasia (nodular regenerative hyperplasia, hamartoma), non-small cell lung carcinoma (NSCLC) (squamous cell lung carcinoma, adenocarcinoma, large cell lung carcinoma), small cell lung carcinoma, thyroid carcinoma, prostate cancer (hormone refractory, androgen independent, androgen dependent, hormone-insensitive), and soft tissue sarcomas (fibrosarcoma, malignant fibrous histiocytoma, dermatofibrosarcoma, liposarcoma, rhabdomyosarcoma leiomyosarcoma, hemangiosarcoma, synovial sarcoma, malignant peripheral nerve sheath tumor/neurofibrosarcoma, extra skeletal osteosarcoma).

In some embodiments, a peptide proteasome inhibitor as provided herein, or a pharmaceutical composition comprising the same, can be administered to treat multiple myeloma in a patient. For example, multiple myeloma can include refractory and/or refractory multiple myeloma or newly diagnosed multiple myeloma.

Many tumors of the hematopoietic and lymphoid tissues are characterized by an increase in cell proliferation, or a particular type of cell. The chronic myeloproliferative diseases (CMPDs) are clonal hematopoietic stem cell disorders characterized by proliferation in the bone marrow of one or more of the myeloid lineages, resulting in increased numbers of granulocytes, red blood cells and/or platelets in the peripheral blood. As such, use of a proteasome inhibitor for the treatment of such diseases is attractive and being examined (Cilloni et al., Haematologica (2007) 92:1124-1229). CMPD can include chronic myelogenous leukemia, chronic neutrophilic leukemia, chronic eosinophilic leukemia, polycythemia vera, chronic idiopathic myelofibrosis, essential thrombocythemia and unclassifiable chronic myeloproliferative disease. Provided herein is a method of treating CMPD comprising administering to a patient in need of such treatment an effective amount of the proteasome inhibitor compound disclosed herein.

Myelodysplastic/myeloproliferative diseases, such as chronic myelomonocytic leukemia, atypical chronic myeloid leukemia, juvenile myelomonocytic leukemia and unclassifiable myelodysplastic/myeloproliferative disease, are characterized by hypercellularity of the bone marrow due to proliferation in one or more of the myeloid lineages. Inhibiting the proteasome with a composition described herein, can serve to treat these myelodysplastic/myeloproliferative diseases by providing a patient in need of such treatment an effective amount of the composition.

Myelodysplastic syndromes (MDS) refer to a group of hematopoietic stem cell disorders characterized by dysplasia and ineffective hematopoiesis in one or more of the major myeloid cell lines. Targeting NF-kB with a proteasome inhibitor in these hematologic malignancies induces apoptosis, thereby killing the malignant cell (Braun et al. Cell Death and Differentiation (2006) 13:748-758). Further provided herein is a method to treat MDS comprising administering to a patient in need of such treatment an effective amount of a compound provided herein. MDS includes refractory anemia, refractory anemia with ringed side oblasts, refractory cytopenia with multilineage dysplasia, refractory anemia with excess blasts, unclassifiable myelodysplastic syndrome and myelodysplastic syndrome associated with isolated del (5q) chromosome abnormality.

Mastocytosis is a proliferation of mast cells and their subsequent accumulation in one or more organ systems, mastocytosis includes, but is not limited to, cutaneous mastocytosis, indolent systemic mastocytosis (ISM), systemic mastocytosis with associated clonal hematological non-mast-cell-lineage disease (SM-AHNMD), aggressive systemic mastocytosis (ASM), mast cell leukemia (MCL), mast cell sarcoma (MCS) and extracutaneous mastocytoma. Further provided herein is a method to treat mastocytosis comprising administering an effect amount of the compound disclosed herein to a patient diagnosed with mastocytosis.

The proteasome regulates NF-κB, which in turn regulates genes involved in the immune and inflammatory response. For example, NF-κB is required for the expression of the immunoglobulin light chain k gene, the IL-2 receptor α-chain gene, the class I major histocompatibility complex gene, and a number of cytokine genes encoding, for example, IL-2, IL-6, granulocyte colony-stimulating factor, and IFN-β (Palombella et al., Cell (1994) 78:773-785). Thus, provided herein are methods of affecting the level of expression of IL-2, MHC-I, IL-6, TNFα, IFN-β or any of the other previously-mentioned proteins, each method comprising administering to a patient an effective amount of a proteasome inhibitor composition disclosed herein.

Also provided herein is a method of treating an autoimmune disease in a patient comprising administering a therapeutically effective amount of the compound described herein. An “autoimmune disease” herein is a disease or disorder arising from and directed against an individual's own tissues. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type I diabetes mellitus or insulin dependent diabetes mellitus); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Beheet disease; giant cell arteritis; immune complex nephritis; IgA nephropathy; IgM polyneuropathies; immune thrombocytopenia purpura (ITP) or autoimmune thrombocytopenia.

The immune system screens for autologous cells that are virally infected, have undergone oncogenic transformation or present unfamiliar peptides on their surface. Intracellular proteolysis generates small peptides for presentation to T-lymphocytes to induce MHC class I-mediated immune responses. Thus, provided herein is a method of using a proteasome inhibitor provided herein as an immunomodulatory agent for inhibiting or altering antigen presentation in a cell, comprising exposing the cell (or administering to a patient) to the compound described herein. Specific embodiments include a method of treating graft or transplant-related diseases, such as graft-versus-host disease or host versus-graft disease in a patient, comprising administering a therapeutically effective amount of the compound described herein. The term “graft” as used herein refers to biological material derived from a donor for transplantation into a recipient. Grafts include such diverse material as, for example, isolated cells such as islet cells; tissue such as the amniotic membrane of a newborn, bone marrow, hematopoietic precursor cells, and ocular tissue, such as corneal tissue; and organs such as skin, heart, liver, spleen, pancreas, thyroid lobe, lung, kidney, tubular organs (e.g., intestine, blood vessels, or esophagus). The tubular organs can be used to replace damaged portions of esophagus, blood vessels, or bile duct. The skin grafts can be used not only for burns, but also as a dressing to damaged intestine or to close certain defects such as diaphragmatic hernia. The graft is derived from any mammalian source, including human, whether from cadavers or living donors. In some cases, the donor and recipient are the same patient. In some embodiments, the graft is bone marrow or an organ such as heart and the donor of the graft and the host are matched for HLA class II antigens.

Histiocytic and dendritic cell neoplasms are derived from phagocytes and accessory cells, which have major roles in the processing and presentation of antigens to lymphocytes. Depleting the proteasome content in dendritic cells has been shown to alter their antigen-induced responses (Chapatte et al. Cancer Res. (2006) 66:5461-5468).

In some embodiments, the cyclodextrin free pharmaceutical formulation or kit as provided herein can be administered to a patient with histiocytic or dendritic cell neoplasm. Histiocytic and dendritic cell neoplasms include histiocytic sarcoma, Langerhans cell histiocytosis, Langerhans cell sarcoma, interdigitating dendritic cell sarcoma/tumor, follicular dendritic cell sarcoma/tumor and non-specified dendritic cell sarcoma.

Inhibition of the proteasome has been shown to be beneficial to treat diseases whereby a cell type is proliferating and immune disorders; thus, in some embodiments, the treatment of lymphoproliferative diseases (LPD) associated with primary immune disorders (PID) is provided comprising administering an effective amount of the disclosed compound to a patient in need thereof. The most common clinical settings of immunodeficiency associated with an increased incidence of lymphoproliferative disorders, including B-cell and T-cell neoplasms and lymphomas, are primary immunodeficiency syndromes and other primary immune disorders, infection with the human immunodeficiency virus (HIV), iatrogenic immunosuppression in patients who have received solid organ or bone marrow allografts, and iatrogenis immunosuppression associated with methotrexate treatment. Other PIDs commonly associated with LPDs, but not limited to, are ataxia telangiectasia (AT), Wiskott-Aldrich syndrome (WAS), common variable immunodeficiency (CVID), severe combined immunodeficiency (SCID), X-linked lymphoproliferative disorder (XLP), Nijmegen breakage syndrome (NBS), hyper-IgM syndrome, and autoimmune lymphoproliferative syndrome (ALPS).

Proteasome inhibition has also been associated with inhibition of NF-κB activation and stabilization of p53 levels. Thus, compositions provided herein may also be used to inhibit NF-κB activation and stabilize p53 levels in cell culture. Since NF-κB is a key regulator of inflammation, it is an attractive target for anti-inflammatory therapeutic intervention. Thus, compositions provided herein may be useful for the treatment of conditions associated with inflammation, including, but not limited to COPD, psoriasis, asthma, bronchitis, emphysema, and cystic fibrosis.

The disclosed compositions can be used to treat conditions mediated directly by the proteolytic function of the proteasome such as muscle wasting or mediated indirectly via proteins which are processed by the proteasome such as NF-κB. The proteasome participates in the rapid elimination and post-translational processing of proteins (e.g., enzymes) involved in cellular regulation (e.g., cell cycle, gene transcription, and metabolic pathways), intercellular communication, and the immune response (e.g., antigen presentation). Specific examples discussed below include β-amyloid protein and regulatory proteins such as cyclins and transcription factor NF-κB.

In some embodiments, a composition provided herein is useful for the treatment of neurodegenerative diseases and conditions, including, but not limited to, stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDS dementia complex, axonomy, diabetic neuropathy, Parkinson's disease, Huntington's disease, multiple sclerosis, bacterial, parasitic, fungal, and viral meningitis, encephalitis, vascular dementia, multi-infarct dementia, Lewy body dementia, frontal lobe dementia such as Pick's disease, subcortical dementias (such as Huntington or progressive supranuclear palsy), focal cortical atrophy syndromes (such as primary aphasia), metabolic-toxic dementias (such as chronic hypothyroidism or B12 deficiency), and dementias caused by infections (such as syphilis or chronic meningitis).

Alzheimer's disease is characterized by extracellular deposits of β-amyloid protein (β-AP) in senile plaques and cerebral vessels. β-AP is a peptide fragment of 39 to 42 amino acids derived from an amyloid protein precursor (APP). At least three isoforms of APP are known (695, 751, and 770 amino acids). Alternative splicing of mRNA generates the isoforms; normal processing affects a portion of the β-AP sequence, thereby preventing the generation of β-AP. It is believed that abnormal protein processing by the proteasome contributes to the abundance of β-AP in the Alzheimer brain. The APP-processing enzyme in rats contains about ten different subunits (22 kDa-32 kDa). The 25 kDa subunit has an N-terminal sequence of X-Gln-Asn-Pro-Met-X-Thr-Gly-Thr-Ser, which is identical to the β-subunit of human macropain (Kojima, S. et al., Fed. Eur. Biochem. Soc., (1992) 304:57-60). The APP-processing enzyme cleaves at the Gln15-Lys16 bond; in the presence of calcium ion, the enzyme also cleaves at the Met-1-Asp1 bond, and the Asp1-Ala2 bonds to release the extracellular domain of β-AP.

One embodiment, therefore, is a method of treating Alzheimer's disease, including administering to a patient an effective amount of a composition provided herein. Such treatment includes reducing the rate of β-AP processing, reducing the rate of β-AP plaque formation, reducing the rate of β-AP generation, and reducing the clinical signs of Alzheimer's disease.

Also provided herein are methods of treating cachexia and muscle-wasting diseases. The proteasome degrades many proteins in maturing reticulocytes and growing fibroblasts. In cells deprived of insulin or serum, the rate of proteolysis nearly doubles.

Inhibiting the proteasome reduces proteolysis, thereby reducing both muscle protein loss and the nitrogenous load on kidneys or liver. Peptide proteasome inhibitors as provided herein are useful for treating conditions such as cancer, chronic infectious diseases, fever, muscle disuse (atrophy) and denervation, nerve injury, fasting, renal failure associated with acidosis, and hepatic failure. See, e.g., Goldberg, U.S. Pat. No. 5,340,736. Methods of treatment include: reducing the rate of muscle protein degradation in a cell; reducing the rate of intracellular protein degradation; reducing the rate of degradation of p53 protein in a cell; and inhibiting the growth of p53-related cancers. Each of these methods includes contacting a cell (in vivo or in vitro, e.g., a muscle in a patient) with an effective amount of a pharmaceutical composition disclosed herein.

Fibrosis is the excessive and persistent formation of scar tissue resulting from the hyperproliferative growth of fibroblasts and is associated with activation of the TGF-β signaling pathway. Fibrosis involves extensive deposition of extracellular matrix and can occur within virtually any tissue or across several different tissues. Normally, the level of intracellular signaling protein (Smad) that activate transcription of target genes upon TGF-β stimulation is regulated by proteasome activity. However, accelerated degradation of the TGF-β signaling components has been observed in cancers and other hyperproliferative conditions. Thus, in certain embodiments, a method for treating hyperproliferative conditions such as diabetic retinopathy, macular degeneration, diabetic nephropathy, glomerulosclerosis, IgA nephropathy, cirrhosis, biliary atresia, congestive heart failure, scleroderma, radiation-induced fibrosis, and lung fibrosis (idiopathic pulmonary fibrosis, collagen vascular disease, sarcoidosis, interstitial lung diseases and extrinsic lung disorders) is provided. The treatment of burn victims is often hampered by fibrosis, thus, in some embodiments an inhibitor provided herein may be administered by topical or systemic administration to treat burns. Wound closure following surgery is often associated with disfiguring scars, which may be prevented by inhibition of fibrosis. Thus, in certain embodiments, a method for the prevention or reduction of scarring is provided herein.

Another protein processed by the proteasome is NF-κB, a member of the Rel protein family. The Rel family of transcriptional activator proteins can be divided into two groups. The first group requires proteolytic processing, and includes p50 (NF-κB1, 105 kDa) and p52 (NF-κ2, 100 kDa). The second group does not require proteolytic processing, and includes p65 (RelA, Rel (c-Rel), and RelB). Both homo- and heterodimers can be formed by Rel family members; NF-κB, for example, is a p50-p65 heterodimer. After phosphorylation and ubiquitination of IκB and p105, the two proteins are degraded and processed, respectively, to produce active NF-κB which translocates from the cytoplasm to the nucleus. Ubiquitinated p105 is also processed by purified proteasomes (Palombella et al., Cell (1994) 78:773-785). Active NF-κB forms a stereospecific enhancer complex with other transcriptional activators and, e.g., HMG I(Y), inducing selective expression of a particular gene.

NF-κB regulates genes involved in the immune and inflammatory response, and mitotic events. For example, NF-κB is required for the expression of the immunoglobulin light chain k gene, the IL-2 receptor α-chain gene, the class I major histocompatibility complex gene, and a number of cytokine genes encoding, for example, IL-2, IL-6, granulocyte colony-stimulating factor, and IFN-β (Palombella et al., Cell (1994) 78:773-785). Some embodiments include methods of affecting the level of expression of IL-2, MHC-I, IL-6, TNFα, IFN-β, or any of the other previously-mentioned proteins, each method including administering to a patient an effective amount of a composition disclosed herein. Complexes including p50 are rapid mediators of acute inflammatory and immune responses (Thanos, D. and Maniatis, T., Cell (1995) 80:529-532).

NF-κB also participates in the expression of the cell adhesion genes that encode E-selectin, P-selectin, ICAM, and VCAM-1 (Collins, T., Lab. Invest. (1993) 68:499-508). In some embodiments, a method for inhibiting cell adhesion (e.g., cell adhesion mediated by E-selectin, P-selectin, ICAM, or VCAM-1) is provided, including contacting a cell with (or administering to a patient) an effective amount of a pharmaceutical composition disclosed herein.

Ischemia and reperfusion injury results in hypoxia, a condition in which there is a deficiency of oxygen reaching the tissues of the body. This condition causes increased degradation of Iκ-Bα, thereby resulting in the activation of NF-κB. It has been demonstrated that the severity of injury resulting in hypoxia can be reduced with the administration of a proteasome inhibitor. Thus, provided herein is a method of treating an ischemic condition or reperfusion injury comprising administering to a patient in need of such treatment an effective amount of a compound disclosed herein. Examples of such conditions or injuries include, but are not limited to, acute coronary syndrome (vulnerable plaques), arterial occlusive disease (cardiac, cerebral, peripheral arterial and vascular occlusions), atherosclerosis (coronary sclerosis, coronary artery disease), infarctions, heart failure, pancreatitis, myocardial hypertrophy, stenosis, and restenosis.

NF-κB also binds specifically to the HIV-enhancer/promoter. When compared to the Nef of mac239, the HIV regulatory protein Nef of pbj14 differs by two amino acids in the region which controls protein kinase binding. It is believed that the protein kinase signals the phosphorylation of IκB, triggering IκB degradation through the ubiquitin-proteasome pathway. After degradation, NF-κB is released into the nucleus, thus enhancing the transcription of HIV (Cohen, J., Science, (1995) 267:960). Provided herein is a method for inhibiting or reducing HIV infection in a patient, and a method for decreasing the level of viral gene expression, each method including administering to the patient an effective amount of a composition disclosed herein.

Viral infections contribute to the pathology of many diseases. Heart conditions such as ongoing myocarditis and dilated cardiomyopathy have been linked to the coxsackievirus B3. In a comparative whole-genome microarray analyses of infected mouse hearts, specific proteasome subunits were uniformly up-regulated in hearts of mice which developed chronic myocarditis (Szalay et al, Am J Pathol 168:1542-52, 2006). Some viruses utilize the ubiquitin-proteasome system in the viral entry step where the virus is released from the endosome into the cytosol. The mouse hepatitis virus (MHV) belongs to the Coronaviridae family, which also includes the severe acute respiratory syndrome (SARS) coronvirus. Yu and Lai (J Virol 79:644-648, 2005) demonstrated that treatment of cells infected with MHV with a proteasome inhibitor resulted in a decrease in viral replication, correlating with reduced viral titer as compared to that of untreated cells. The human hepatitis B virus (HBV), a member of the Hepadnaviridae virus family, likewise requires virally encoded envelop proteins to propagate. Inhibiting the proteasome degradation pathway causes a significant reduction in the amount of secreted envelope proteins (Simsek et al, J Virol 79:12914-12920, 2005). In addition to HBV, other hepatitis viruses (A, C, D and E) may also utilize the ubiquitin-proteasome degradation pathway for secretion, morphogenesis and pathogenesis. Accordingly, in certain embodiments, a method for treating viral infection, such as SARS or hepatitis A, B, C, D and E, is provided comprising contacting a cell with (or administering to a patient) an effective amount of the compound disclosed herein.

Overproduction of lipopolysaccharide (LPS)-induced cytokines such as TNFα is considered to be central to the processes associated with septic shock. Furthermore, it is generally accepted that the first step in the activation of cells by LPS is the binding of LPS to specific membrane receptors. The α- and β-subunits of the 20S proteasome complex have been identified as LPS-binding proteins, suggesting that the LPS-induced signal transduction may be an important therapeutic target in the treatment or prevention of sepsis (Qureshi, N. et al., J. Immun. (2003) 171:1515-1525). Therefore, in certain embodiments, compositions as provided herein may be used for the inhibition of TNFα to prevent and/or treat septic shock.

Intracellular proteolysis generates small peptides for presentation to T-lymphocytes to induce MHC class I-mediated immune responses. The immune system screens for autologous cells that are virally infected or have undergone oncogenic transformation. One embodiment is a method for inhibiting antigen presentation in a cell, including exposing the cell to a composition described herein. A further embodiment is a method for suppressing the immune system of a patient (e.g., inhibiting transplant rejection, allergy, asthma), including administering to the patient an effective amount of a composition described herein. Compositions provided herein can also be used to treat autoimmune diseases such as lupus, rheumatoid arthritis, multiple sclerosis, and inflammatory bowel diseases such as ulcerative colitis and Crohn's disease.

Another embodiment is a method for altering the repertoire of antigenic peptides produced by the proteasome or other Ntn with multicatalytic activity. For example, if the PGPH activity of 20S proteasome is selectively inhibited, a different set of antigenic peptides will be produced by the proteasome and presented in MHC molecules on the surfaces of cells than would be produced and presented either without any enzyme inhibition, or with, for example, selective inhibition of chymotrypsin-like activity of the proteasome.

Certain proteasome inhibitors block both degradation and processing of ubiquitinated NF-κB in vitro and in vivo. Proteasome inhibitors also block IκB-α degradation and NF-κB activation (Palombella, et al. Cell (1994) 78:773-785; and Traenckner, et al., EMBO J. (1994) 13:5433-5441). In some embodiments, a method for inhibiting IκB-α degradation is provided, including contacting the cell with a composition described herein. A further embodiment is a method for reducing the cellular content of NF-κB in a cell, muscle, organ, or patient, including contacting the cell, muscle, organ, or patient with a composition described herein.

Other eukaryotic transcription factors that require proteolytic processing include the general transcription factor TFIIA, herpes simplex virus VP16 accessory protein (host cell factor), virus-inducible IFN regulatory factor 2 protein, and the membrane-bound sterol regulatory element-binding protein 1.

Further provided herein are methods for affecting cyclin-dependent eukaryotic cell cycles, including exposing a cell (in vitro or in vivo) to a composition disclosed herein. Cyclins are proteins involved in cell cycle control. The proteasome participates in the degradation of cyclins. Examples of cyclins include mitotic cyclins, G1 cyclins, and cyclin B. Degradation of cyclins enables a cell to exit one cell cycle stage (e.g., mitosis) and enter another (e.g., division). It is believed all cyclins are associated with p34cdc2 protein kinase or related kinases. The proteolysis targeting signal is localized to amino acids 42-RAALGNISEN-50 (destruction box). There is evidence that cyclin is converted to a form vulnerable to a ubiquitin ligase or that a cyclin-specific ligase is activated during mitosis (Ciechanover, A., Cell, (1994) 79:13-21). Inhibition of the proteasome inhibits cyclin degradation, and therefore inhibits cell proliferation, for example, in cyclin-related cancers (Kumatori et al., Proc. Natl. Acad. Sci. USA (1990) 87:7071-7075). Provided herein is a method for treating a proliferative disease in a patient (e.g., cancer, psoriasis, or restenosis), including administering to the patient an effective amount of a composition disclosed herein. Also provided herein is a method for treating cyclin-related inflammation in a patient, including administering to a patient a therapeutically effective amount of a composition described herein.

Additional embodiments include methods for affecting the proteasome-dependent regulation of oncoproteins and methods of treating or inhibiting cancer growth, each method including exposing a cell (in vivo, e.g., in a patient, or in vitro) to a composition disclosed herein. HPV-16 and HPV-18-derived E6 proteins stimulate ATP- and ubiquitin-dependent conjugation and degradation of p53 in crude reticulocyte lysates. The recessive oncogene p53 has been shown to accumulate at the nonpermissive temperature in a cell line with a mutated thermolabile E1. Elevated levels of p53 may lead to apoptosis. Examples of proto-oncoproteins degraded by the ubiquitin system include c-Mos, c-Fos, and c-Jun. One embodiment is a method for treating p53-related apoptosis, including administering to a patient an effective amount of a composition disclosed herein.

In another embodiment, the disclosed compositions are useful for the treatment of a parasitic infection, such as infections caused by protozoan parasites. The proteasome of these parasites is considered to be involved primarily in cell differentiation and replication activities (Paugam et al., Trends Parasitol. 2003, 19(2): 55-59). Furthermore, entamoeba species have been shown to lose encystation capacity when exposed to proteasome inhibitors (Gonzales, et al., Arch. Med. Res. 1997, 28, Spec No: 139-140). In certain such embodiments, the disclosed compositions are useful for the treatment of parasitic infections in humans caused by a protozoan parasite selected from Plasmodium sps. (including P. falciparum, P. vivax, P. malariae, and P. ovale, which cause malaria), Trypanosoma sps. (including T. cruzi, which causes Chagas' disease, and T. brucei which causes African sleeping sickness), Leishmania sps. (including L. amazonesis, L. donovani, L. infantum, L. mexicana, etc.), Pneumocystis carinii (a protozoan known to cause pneumonia in AIDS and other immunosuppressed patients), Toxoplasma gondii, Entamoeba histolytica, Entamoeba invadens, and Giardia lamblia. In certain embodiments, the disclosed compositions are useful for the treatment of parasitic infections in animals and livestock caused by a protozoan parasite selected from Plasmodium hermani, Cryptosporidium sps., Echinococcus granulosus, Eimeria tenella, Sarcocystis neurona, and Neurospora crassa. Other compounds useful as proteasome inhibitors in the treatment of parasitic diseases are described in WO 98/10779, which is incorporated herein in its entirety.

In certain embodiments, the disclosed compositions inhibit proteasome activity irreversibly in a parasite. Such irreversible inhibition has been shown to induce shutdown in enzyme activity without recovery in red blood cells and white blood cells. In certain such embodiments, the long half-life of blood cells may provide prolonged protection with regard to therapy against recurring exposures to parasites. In certain embodiments, the long half-life of blood cells may provide prolonged protection with regard to chemoprophylaxis against future infection.

Prokaryotes have what is equivalent to the eukaryote 20S proteasome particle. Albeit, the subunit composition of the prokaryote 20S particle is simpler than that of eukaryotes, it has the ability to hydrolyze peptide bonds in a similar manner. For example, the nucleophilic attack on the peptide bond occurs through the threonine residue on the N-terminus of the β-subunits. In some embodiments, a method of treating prokaryotic infections is provided, comprising administering to a patient an effective amount of the proteasome inhibitor composition disclosed herein. Prokaryotic infections may include diseases caused by either mycobacteria (such as tuberculosis, leprosy or Buruli Ulcer) or archaebacteria.

It has also been demonstrated that inhibitors that bind to the 20S proteasome stimulate bone formation in bone organ cultures. Furthermore, when such inhibitors have been administered systemically to mice, certain proteasome inhibitors increased bone volume and bone formation rates over 70% (Garrett, I. R. et al., J. Clin. Invest. (2003) 111: 1771-1782), therefore suggesting that the ubiquitin-proteasome machinery regulates osteoblast differentiation and bone formation. Therefore, the disclosed compositions may be useful in the treatment and/or prevention of diseases associated with bone loss, such as osteoporosis.

Provided herein is a method for treating a disease or condition selected from cancer, autoimmune disease, graft or transplant-related condition, neurodegenerative disease, fibrotic-associated condition, ischemic-related conditions, infection (viral, parasitic or prokaryotic) and diseases associated with bone loss, comprising administering a proteasome inhibitor as provided herein. For example, a compound of formula (5).

Bone tissue is an excellent source for factors which have the capacity for stimulating bone cells. Thus, extracts of bovine bone tissue contain not only structural proteins which are responsible for maintaining the structural integrity of bone, but also biologically active bone growth factors which can stimulate bone cells to proliferate. Among these latter factors are a recently described family of proteins called bone morphogenetic proteins (BMPs). All of these growth factors have effects on other types of cells, as well as on bone cells, including Hardy, M. H., et al., Trans Genet (1992) 8:55-61 describes evidence that bone morphogenetic proteins (BMPs), are differentially expressed in hair follicles during development. Harris, S. E., et al., J Bone Miner Res (1994) 9:855-863 describes the effects of TGF-β on expression of BMP-2 and other substances in bone cells. BMP-2 expression in mature follicles also occurs during maturation and after the period of cell proliferation (Hardy, et al. (1992, supra). Thus, compounds provided herein may also be useful for hair follicle growth stimulation.

Finally, the disclosed compositions are also useful as diagnostic agents (e.g., in diagnostic kits or for use in clinical laboratories) for screening for proteins (e.g., enzymes, transcription factors) processed by Ntn hydrolases, including the proteasome. The disclosed compositions are also useful as research reagents for specifically binding the X/MB1 subunit or α-chain and inhibiting the proteolytic activities associated with it. For example, the activity of (and specific inhibitors of) other subunits of the proteasome can be determined.

Most cellular proteins are subject to proteolytic processing during maturation or activation. Enzyme inhibitors disclosed herein can be used to determine whether a cellular, developmental, or physiological process or output is regulated by the proteolytic activity of a particular Ntn hydrolase. One such method includes obtaining an organism, an intact cell preparation, or a cell extract; exposing the organism, cell preparation, or cell extract to a composition disclosed herein; exposing the compound-exposed organism, cell preparation, or cell extract to a signal, and monitoring the process or output. The high selectivity of the compounds disclosed herein permits rapid and accurate elimination or implication of the Ntn (for example, the 20S proteasome) in a given cellular, developmental, or physiological process.

### Administration

Compositions prepared as described herein can be administered in various forms, depending on the disorder to be treated and the age, condition, and body weight of the patient, as is well known in the art. For example, where the compositions are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means in conjunction with the methods described herein, and, if desired, the active ingredient may be mixed with any conventional additive or excipient, such as a binder, a disintegrating agent, a lubricant, a corrigent, a solubilizing agent, a suspension aid, an emulsifying agent, or a coating agent in addition to a cyclodextrin and a buffer. Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses. The amount of active ingredient which can be combined with a carrier material to produce a single dosage form will generally be that amount of the compound which produces a therapeutic effect. In general, compositions intended for parenteral use (e.g., intravenous, subcutaneous injection) include a substituted cyclodextrin. Compositions administered via other routes, particularly the oral route, include a substituted or unsubstituted cyclodextrin.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the patient and adjusting the dosage and/or timing.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions provided herein are non-pyrogenic, i.e., do not induce significant temperature elevations when administered to a patient.

The term “pharmaceutically acceptable salt” refers to the relatively non-toxic, inorganic and organic acid addition salts of the inhibitor(s). These salts can be prepared in situ during the final isolation and purification of the inhibitor(s), or by separately reacting a purified peptide proteasome inhibitor in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66:1-19.)

In some embodiments, the peptide proteasome inhibitors provided herein may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of an inhibitor(s) as an active ingredient. A composition may also be administered as a bolus, electuary, or paste.

In solid dosage forms for oral administration (capsules, tablets, pills, dragees, powders, granules, and the like), the active ingredient is mixed with one or more pharmaceutically acceptable carriers, such as sodium citrate or dicalcium phosphate, and/or any of the following: (1) fillers or extenders, such as starches, cyclodextrins, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to inhibitor(s), excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to an inhibitor(s), excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A peptide proteasome inhibitor can be administered by aerosol. This is accomplished by preparing an aqueous aerosol, liposomal preparation, or solid particles containing the composition. A nonaqueous (e.g., fluorocarbon propellant) suspension could be used. In some embodiments, sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, sorbitan esters, oleic acid, lecithin, amino acids such as glycine, buffers, salts, sugars, or sugar alcohols. Aerosols generally are prepared from isotonic solutions.

Transdermal patches have the added advantage of providing controlled delivery of an inhibitor(s) to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions suitable for parenteral administration comprise one or more peptide proteasome inhibitors in combination with one or more pharmaceutically acceptable sterile aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions provided herein include water for injection (e.g., sterile water for injection), ethanol, polyols (such as glycerol, propylene glycol, polyethylene glycol, and the like), buffer (such as citrate buffer), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Pharmaceutical compositions typically include a pharmaceutically acceptable carrier. As used herein the language “pharmaceutically acceptable carrier” includes buffer, sterile water for injection, solvents, dispersion media, coatings, antibacterial and antifungal agents, isotonic and absorption delaying agents, and the like, compatible with pharmaceutical administration. In some embodiments, a pharmaceutically acceptable carrier is a buffer (e.g., citrate buffer). In some embodiments, a pharmaceutically acceptable carrier is sterile water for injection. In some embodiments, a pharmaceutically acceptable carrier comprises citric acid.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of agents may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. In some embodiments, administration is oral.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The phrases “systemic administration,” “administered systemically,” “peripheral administration” and “administered peripherally” as used herein mean the administration of a ligand, drug, or other material other than directly into the central nervous system, such that it enters the patient's system and thus, is subject to metabolism and other like processes, for example, subcutaneous administration.

The peptide proteasome inhibitors described herein may be administered to humans and other animals for therapy by any suitable route of administration, including orally, nasally, as by, for example, a spray, rectally, intravaginally, parenterally, intracistemally, and topically, as by powders, ointments or drops, including buccally and sublingually.

Regardless of the route of administration selected, a peptide proteasome inhibitor, which may be used in a suitable hydrated form, and/or the pharmaceutical compositions provided herein, is formulated into a pharmaceutically acceptable dosage form by conventional methods known to those of skill in the art.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The concentration of a disclosed compound in a pharmaceutically acceptable mixture will vary depending on several factors, including the dosage of the compound to be administered, the pharmacokinetic characteristics of the compound(s) employed, and the route of administration. In general, the compositions provided herein may be provided in an aqueous solution containing about 0.1-10% w/v of a compound disclosed herein, among other substances, for parenteral administration. Typical dose ranges are from about 0.01 to about 50 mg/kg of body weight per day, given in 1-4 divided doses. Each divided dose may contain the same or different compounds. The dosage will be an effective amount depending on several factors including the overall health of a patient, and the formulation and route of administration of the selected compound(s).

In another embodiment, the pharmaceutical composition is an oral solution or a parenteral solution. Another embodiment is a freeze-dried preparation that can be reconstituted prior to administration. As a solid, this formulation may also include tablets, capsules or powders.

Also provided herein is a conjoint therapy wherein one or more other therapeutic agents are administered with a peptide proteasome inhibitor or a pharmaceutical composition comprising a peptide proteasome inhibitor. Such conjoint treatment may be achieved by way of the simultaneous, sequential, or separate dosing of the individual components of the treatment.

In certain embodiments, the cyclodextrin free pharmaceutical formulation or kit as provided herein can be conjointly administered with one or more other proteasome inhibitor(s).

In certain embodiments, the cyclodextrin free pharmaceutical formulation or kit as provided herein can be conjointly administered with one or more chemotherapeutics. Suitable chemotherapeutics may include, natural products such as vinca alkaloids (i.e. vinblastine, vincristine, and vinorelbine), taxanes (e.g., docetaxel, paclitaxel, e.g., docetaxel), epidipodophyllotoxins (i.e. etoposide, teniposide), antibiotics (dactinomycin (actinomycin D) daunorubicin, doxorubicin and idarubicin; e.g., doxorubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, ifosphamide, cyclophosphamide and analogs, melphalan, chlorambucil, e.g., melphalan), ethylenimines and methylmelamines (hexaamethylmelaamine and thiotepa), alkyl sulfonates (busulfan), nitrosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine); aromatase inhibitors (anastrozole, exemestane, and letrozole); platinum coordination complexes (cisplatin, carboplatin), procarbazine, hydroxyurea, mitotane, aminoglutethimide; DNA binding/Cytotoxic agents (e.g., Zalypsis); histone deacetylase (HDAC) inhibitors (e.g., trichostatin, sodium butyrate, apicidan, suberoyl anilide hydroamic acid (SAHA (Vorinostat)), trichostatin A, depsipeptide, apicidin, A-161906, scriptaid, PXD-101, CHAP, butyric acid, depudecin, oxamflatin, phenylbutyrate, valproic acid, MS275 (N-(2-Aminophenyl)-4-[N-(pyridine-3-ylmethoxy-carbonyl) aminomethyl]benzamide), LAQ824/LBH589, CI994, MGCD0103, ACY-1215, Panobinostat); hormones (i.e. estrogen) and hormone agonists such as leutinizing hormone releasing hormone (LHRH) agonists (goserelin, leuprolide and triptorelin). Other chemotherapeutic agents may include mechlorethamine, camptothecin, ifosfamide, tamoxifen, raloxifene, gemcitabine, navelbine, or any analog or derivative variant of the foregoing.

In certain embodiments, the cyclodextrin free pharmaceutical formulation or kit as provided herein can be conjointly administered with one or more histone deacetylase (HDAC) inhibitors (e.g., trichostatin, sodium butyrate, apicidan, suberoyl anilide hydroamic acid (“SAHA” (Vorinostat)), trichostatin A, depsipeptide, apicidin, A-161906, scriptaid, PXD-101, CHAP, butyric acid, depudecin, oxamflatin, phenylbutyrate, valproic acid, MS275 (N-(2-Aminophenyl)-4-[N-(pyridine-3-ylmethoxy-carbonyl) aminomethyl]benzamide), LAQ824/LBH589, CI994, MGCD0103, ACY-1215, Panobinostat; e.g., SAHA, ACY-1215, Panobinostat).

In certain embodiments, the cyclodextrin free pharmaceutical formulation or kit as provided herein can be conjointly administered with one or more nitrogen mustards (mechlorethamine, ifosphamide, cyclophosphamide and analogs, melphalan, chlorambucil, e.g., melphalan).

In certain embodiments, the cyclodextrin free pharmaceutical formulation or kit as provided herein can be conjointly administered with one or more DNA binding/Cytotoxic agents (e.g., Zalypsis).

In certain embodiments, the cyclodextrin free pharmaceutical formulation or kit as provided herein can be conjointly administered with one or more taxanes (e.g., docetaxel, paclitaxel, e.g., docetaxel).

In certain embodiments, the cyclodextrin free pharmaceutical formulation or kit as provided herein can be conjointly administered with one or more antibiotics (dactinomycin (actinomycin D) daunorubicin, doxorubicin and idarubicin; e.g., doxorubicin).

In some embodiments, the cyclodextrin free pharmaceutical formulation or kit as provided herein can be conjointly administered with one or more cytokines. Cytokines include, but are not limited to, Interferon-γ, -α, and -β, Interleukins 1-8, 10 and 12, Granulocyte Monocyte Colony-Stimulating factor (GM-CSF), TNF-α and -β, and TGF-β.

In some embodiments, the cyclodextrin free pharmaceutical formulation or kit provided herein can be conjointly administered with one or more steroids. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts and/or derivatives thereof (e.g., hydrocortisone, dexamethasone, methylprednisolone and prednisolone; e.g., dexamethasone).

In certain embodiments, the cyclodextrin free pharmaceutical formulation or kit provided herein can be conjointly administered with dexamethasone. In certain embodiments, conjoint therapy includes the dosing regimens provided on the KYPROLIS label, e.g.,

1. KYPROLIS is administered intravenously over 2 to 10 minutes, on two consecutive days, each week for three weeks (Days 1, 2, 8, 9, 15, and 16), followed by a 12-day rest period (Days 17 to 28). Each 28-day period is considered one treatment cycle (Table A).

In Cycle 1, KYPROLIS is administered at a dose of 20 mg/m². If tolerated in Cycle 1, the dose should be escalated to 27 mg/m² beginning in Cycle 2 and continued at 27 mg/m² in subsequent cycles. Treatment may be continued until disease progression or until unacceptable toxicity occurs.

The dose is calculated using the patient's actual body surface area at baseline. Patients with a body surface area greater than 2.2 m² should receive a dose based upon a body surface area of 2.2 m². Dose adjustments do not need to be made for weight changes of less than or equal to 20%.

TABLE A1
KYPROLIS ® Dosage Regimen for Patients with Multiple Myeloma

| | Week 1 | | | Week 2 | | | Week 3 | | | Week 4 |
|---|---|---|---|---|---|---|---|---|---|---|
| | Day | Day | Days | Day | Day | Days | Day | Day | Days | Days |
| | 1 | 2 | 3-7 | 8 | 9 | 10-14 | 15 | 16 | 17-21 | 22-28 |
| | Cycle 1 | | | | | | | | | |
| KYPROLIS | 20 | 20 | No | 20 | 20 | No | 20 | 20 | No | No |
| (20 mg/m²): | | | Dosing | | | Dosing | | | Dosing | Dosing |
| | Cycle 2 and Beyond^{a} | | | | | | | | | |
| KYPROLIS | 27 | 27 | No | 27 | 27 | No | 27 | 27 | No | No |
| (27 mg/m²): | | | Dosing | | | Dosing | | | Dosing | Dosing |

^{a}If previous cycle dosage is tolerated.

2. Hydrate patients to reduce the risk of renal toxicity and of tumor lysis syndrome (TLS) with KYPROLIS treatment. Maintain adequate fluid volume status throughout treatment and monitor blood chemistries closely. Prior to each dose in Cycle 1, give 250 mL to 500 mL of intravenous normal saline or other appropriate intravenous fluid. Give an additional 250 mL to 500 mL of intravenous fluids as needed following KYPROLIS administration. Continue intravenous hydration, as needed, in subsequent cycles. Also monitor patients during this period for fluid overload.

3. Pre-medicate with dexamethasone 4 mg orally or intravenously prior to all doses of KYPROLIS during Cycle 1 and prior to all KYPROLIS doses during the first cycle of dose escalation to 27 mg/m² to reduce the incidence and severity of infusion reactions. Reinstate dexamethasone premedication (4 mg orally or intravenously) if these symptoms develop or reappear during subsequent cycles.

In some embodiments, the cyclodextrin free pharmaceutical formulation or kit as provided herein can be conjointly administered with one or more immunotherapeutic agents. Suitable immunotherapeutic agents may include, but are not limited to, MDR modulators (verapamil, valspordar, biricodar, tariquidar, laniquidar), cyclosporine, pomalidomide, thalidomide, CC-4047 (Actimid), lenalidomide (Revlimid) and monoclonal antibodies. The monoclonal antibodies can be either naked or conjugated such as rituximab, tositumomab, alemtuzumab, epratuzumab, ibritumomab tiuxetan, gemtuzumab ozogamicin, bevacizumab, cetuximab, erlotinib and trastuzumab. In certain embodiments, a pharmaceutical composition provided herein is conjointly administered with lenalidomide (Revlimid).

In some embodiments, the cyclodextrin free pharmaceutical formulation or kit provided herein (e.g., pharmaceutical compositions that include carfilzomib) can be conjointly administered with
(i) one or more of the following:
   one or more second chemotherapeutic agents (e.g., one or more HDAC inhibitors, e.g., SAHA, ACY-1215, Panobinostat; one or more nitrogen mustards e.g., melphalan; one or more DNA binding/cytotoxic agents, e.g., Zylapsis; one or more taxanes, e.g., docetaxel; one or more antibiotics (dactinomycin (actinomycin D) daunorubicin, doxorubicin and idarubicin; e.g., doxorubicin);
   one or more other proteasome inhibitor(s) (e.g., another compound of formulae (1)-(5));
   one or more cytokines;
   one or more immunotherapeutic agents (e.g., Revlimid);
   one or more topoisomerase inhibitors;
   one or more m-TOR inhibitors;
   one or more protein kinase inhibitors (e.g., sorafenib);
   one or more CDK Inhibitors (e.g., Dinaciclib);
   one or more KSP (Eg5) Inhibitors (e.g., Array 520);
   one or more PI13 delta Inhibitors (e.g., GS-1101 PI3K);
   one or more Dual Inhibitor: PI3K delta and gamma Inhibitors (e.g., CAL-130);
   one or more multi-kinase Inhibitors (e.g., TG02);
   one or more PI3K delta Inhibitors (e.g., TGR-1202); and
(ii) one or more steroids (e.g., dexamethasone).

In other embodiments, the cyclodextrin free pharmaceutical formulation or kit provided herein can be conjointly administered with
(i) one of the following:
   one or more second chemotherapeutic agents (e.g., one or more HDAC inhibitors, e.g., SAHA, ACY-1215, Panobinostat; one or more nitrogen mustards e.g., melphalan; one or more DNA binding/cytotoxic agents, e.g., Zylapsis; one or more taxanes, e.g., docetaxel; one or more antibiotics (dactinomycin (actinomycin D) daunorubicin, doxorubicin and idarubicin; e.g., doxorubicin);
   one or more other proteasome inhibitor(s) (e.g., another compound of formulae (1)-(5));
   one or more cytokines;
   one or more immunotherapeutic agents (e.g., Revlimid);
   one or more topoisomerase inhibitors;
   one or more m-TOR inhibitors;
   one or more protein kinase inhibitors (e.g., sorafenib);
   one or more CDK Inhibitors (e.g., Dinaciclib);
   one or more KSP (Eg5) Inhibitors (e.g., Array 520);
   one or more PI13 delta Inhibitors (e.g., GS-1101 PI3K);
   one or more Dual Inhibitor: PI3K delta and gamma Inhibitors (e.g., CAL-130);
   one or more multi-kinase Inhibitors (e.g., TG02);
   one or more PI3K delta Inhibitors (e.g., TGR-1202);
and
(ii) dexamethasone.

### EXAMPLES

### Example 1: Non-Aqueous and Aqueous Solvent Screenings

In the initial solvent screening, three targeted CFZ-API concentrations were used to examine their solubility profiles, as follows:

Non-aqueous phase: (a) 200 mg/ml to 250 mg/ml of CFZ-API target concentration for the water miscible solvent screening; and (b) 20 to 50 mg/ml of CFZ-API target concentration for the co-solvent screening; and Aqueous phase: about 2 mg/ml target CFZ-API final concentration.

### Example 1A. Non-Aqueous Phase Solvent Screening

a. Organic Water Miscible Solvent Screening.

Since CFZ-API is extremely insoluble in water, various organic water miscible solvents were first screened to dissolve CFZ-API drug substance powder. Table 1 shows the visual observations of solubility pattern to meet the initial 200 mg/ml to 250 mg/ml CFZ-API concentration in various water miscible solvents. As shown in Table 1, test solutions Nos. 7 to 11 resulted in CFZ-API insolubility, and therefore only test solutions Nos. 1 to 6 were taken forward to the co-solvent screening.

TABLE 1
Screening of Various Organic Water Miscible Solvents
to Dissolve CFZ-API at High Concentration (≥200 mg/ml)

| | Water Miscible | CFZ-API | | |
|---|---|---|---|---|
| | Solvents (Neat) | Concentration | | Time forCFZ |
| No. | Mixed with CFZ-API | (mg/ml) | Visual Observation | to turn clear |
| 1 | DMSO | ≥200 | Clear | Seconds |
| 2 | NMP | ≥200 | Clear | Seconds |
| 3 | DMSO + NMP | ≥200 | Clear | Seconds |
| 4 | Dimethylacetamide (DMA) | ≥200 | Clear | Seconds |
| 5 | Ethyl Lactate | ≥200 | Clear | Seconds |
| 6 | Benzyl Alcohol | ≥200 | Clear | About 5 Minutes |
| 7 | Benzyl Benzoate | N/A | Opaque yellow; Clumps of | Did not dissolve |
| | | | CFZ-API floating | |
| 8 | Ethyl Oleate | N/A | Opaque | Did not dissolve |
| 9 | Triglycerides | N/A | Opaque | Did not dissolve |
| 10 | Ethyl Lactate + DMA | N/A | Clumps of CFZ-API floating | Did not dissolve |
| 11 | Water containing solution | N/A | Not clear; Clumps of CFZ- | Did not dissolve |
| | (i.e. PBS or dextrose) | | API floating | |

b. Co-Solvents Screening

Co-solvents screening was conducted to further dilute the CFZ-API in the above identified non-aqueous water miscible solution to the second targeted concentration of 20-50 mg/ml. Based on the results in Table 1 above, DMSO or NMP were chosen as the preferred water miscible solvents. Table 2 lists the various co-solvents that were further screened to dilute the CFZ-API previously dissolved in DMSO or NMP water miscible solvents to meet the targeted 20-50 mg/ml CFZ-API concentration. Non-nucleophilic solvents and/or excipients were carefully selected for the screening because CFZ-API contains an epoxy ketone moiety that is sensitive to nucleophilic attack. In addition, parenterally approved solvents/excipients for intravenous injection (IV) and subcutaneous injection (SC) with acceptable concentrations based on FDA injection limits were also carefully selected for the screening.

TABLE 2
Co-solvents screening to further dilute CFZ-
API to a target concentration of 20 to 50 mg/ml

| No. | Co-Solvents* | Visual Observations |
|---|---|---|
| 1 | 75% to 92% PEG400:Ethanol (1:1, w/w) | Clear |
| 2 | 1.2% to 5% Lactic Acid in PEG400:Ethanol | Clear |
| 3 | 1.2% to 5% Maleic Acid in PEG400:Ethanol | Clear |
| 4 | 4.6% Benzokonium Chloride in PEG400:Ethanol | Clear |
| 5 | 1% to 3.3% Protamine Sulfate in PEG400:Ethanol | Clear |
| 6 | 28% to 30% HS Solutol 15 in PEG400:Ethanol | Clear |
| 7 | 32% Sucrose cocoate, PEG400:Ethanol | Clear |
| 8 | 5% Benzoic Acid, PEG400:Ethanol | Clear |
| 9 | 5% Benzenesulfonic Acid, PEG400:Ethanol | Clear |
| 10 | 10% Isopropyl Alcohol, PEG400:Ethanol | Clear |
| 11 | 1% to 5% Citric Acid in PEG400:Ethanol | Clear |
| 12 | 1.2% to 5% Acetic Acid in PEG400:Ethanol | Clear |
| 13 | 5% Ethanolamine in PEG400:Ethanol | Clear |
| 14 | 50% PEG400:Ethanol (1:1), Dextrose, Acetate pH5, water | Precipitated |
| 15 | 50% PEG400:Ethanol (1:1), Dextrose, water | Precipitated |
| 16 | 3% Octanoic Acid, PEG400:Ethanol | Clear but had micelles of |
| | | oil-like properties |
| 17 | 5% Tributyl phosphate, PEG400:Ethanol | Clear but had micelles of |
| | | oil-like properties |
| 18 | 6% to 33% N-acetyl Tryptophan in PEG400:Ethanol | Clear at higher |
| | | concentrations of co-solvent |
| 19 | 3% Octanoic Acid, sucrose, water | Clear but had micelles of |
| | | oil-like properties |
| 20 | 5% Tributyl phosphate, sucrose, water | Clear but had micelles of |
| | | oil-like properties |
| 21 | 32% Sucrose cocoate, sucrose, water | Precipitated |
| 22 | 5% Benzoic Acid, sucrose, water | Precipitated |
| 23 | 5% Benzenesulfonic Acid, sucrose, water | Precipitated |
| 24 | 10% Isopropyl Alcohol, sucrose, water | Precipitated |
| 25 | 5% Lactic Acid in sucrose, water | Precipitated |
| 26 | 5% Maleic Acid in sucrose, water | Precipitated |
| 27 | 5% Citric Acid in sucrose, water | Precipitated |
| 28 | 1% to 5% Acetic Acid in sucrose, water | Precipitated |
| 29 | 4.6% Benzokonium Chloride in sucrose, water | Precipitated |
| 30 | 30% HS Solutol 15 in sucrose, water | Precipitated |
| 31 | 3.3% Protamine Sulfate in sucrose, water | Precipitated |
| 32 | 5% Ethanolamine in sucrose, water | Precipitated |
| 33 | 6% to 33% N-acetyl Tryptophan, sucrose, water | Precipitated |
| 34 | 10% Isopropyl Alcohol, sucrose, water | Precipitated |

*CFZ-API was first dissolved in neat NMP or DMSO to about 200 mg/ml to 250 mg/ml. The co-solvents listed in Table 2 were added to further dilute CFZ-API to about 20 mg/ml to 50 mg/ml.

The sample formulations where the CFZ-API precipitated were expected to have a lower CFZ-API recovery after filtration than those sample formulations that did not precipitate. Placebo buffers of each condition was used to determine the CFZ-API precipitated out of solution rather than any other substance. Visual observations of sample Nos. 14-15 and 21-34 in Table 2 indicated that presence of water caused precipitation of CFZ-API at the targeted concentration range of 20 mg/ml to 50 mg/ml. Sample Nos. 14 to 34 from Table 2 resulted in CFZ-API insolubility and only Sample Nos. 1 to 13 were further investigated in the next aqueous solvent screening.

It was found that lactic acid and maleic acid in combination with PEG400 and ethanol gave maximal solubility as determined through visual observation when diluted to 2 mg/ml with povidone and water to pH 3 which will be discussed on the next section. Moreover, the lactic acid containing formulation was found to have longer term CFZ-API solubility at 2° C. to 8° C. as compared to maleic acid containing formulation which was precipitating after 3 days at 2° C. to 8° C. storage. The lack of lactic acid in the formulation showed precipitation of CFZ-API at pH 3 and therefore lactic acid contributed to lowering the pH as well as acting as a co-solubilizer of CFZ-API.

### Example 1B: Aqueous Phase Solvents Screening

The introduction of water to CFZ-API was conducted in the presence of one or more solubilizers to anticipate better dispersion of the compound within the solution. Although precipitation was observed in all samples at this step, a pH adjustment with methane sulfonic acid was conducted to achieve pH range of 3.0-3.1 to increase solubility. Since most of the samples were turbid even after the pH adjustment, the solutions were filtered on 0.22 μm PES membrane filter to check for CFZ-API solubility on RP-HPLC. Table 3 shows visual observations post pH adjustment and filtration, as well as CFZ-API solubility of the different bulking agents and solubilizers with water that were used in combination of CFZ-API in DMSO, PEG400, Ethanol and lactic acid to reach to 2 mg/ml CFZ-API. The results determine that all PVP-containing formulations (conditions 3 to 8 in Table 3) yielded higher CFZ-API solubility compared to other formulations. Based on solubility evidence from NMP and PVP, we suspect that other molecules with the pyrrolidone ring or similar structure will solubilize CFZ-API.

TABLE 3
Aqueous solvent screening to dissolve CFZ-API* to about 2 mg/ml

| | | | Visual | | |
|---|---|---|---|---|---|
| | | Visual | Observation at | CFZ Solubility | |
| | | Observation | pH 3.0, post- | Concentration | |
| No. | Solubilizers | at pH 3.0 | filtration | post- filtration | |
| 1 | Water | Turbid | Clear | 1.1 | mg/ml |
| 2 | 4.8% Dextrose | Turbid | Clear | 1.0 | mg/ml |
| 3 | 29% PVP 10,000 MW | Clear | Clear | 2.2 | mg/ml |
| 4 | 24% PVP 10,000 MW | Clear | Clear | 1.8 | mg/ml |
| 5 | 24% PVP 12,000 MW | Turbid (less than others) | Clear | 2.0 | mg/ml |
| 6 | 20% PVP 12,000 MW | Turbid | Clear | 2.0 | mg/ml |
| 7 | 10% PVP 12,000 MW | Turbid | Clear | 1.5 | mg/ml |
| 8 | 24% PVP 17,000 MW | Turbid | Clear | 2.0 | mg/ml |
| 9 | 48 mM Mannitol | Turbid | Clear | 1.3 | mg/ml |
| 10 | 96 mM Mannitol | Turbid | Clear | 1.5 | mg/ml |
| 11 | 192 mM Mannitol | Turbid | Clear | 1.4 | mg/ml |
| 12 | 96 mM Glycine | Turbid | Clear | 0.8 | mg/ml |
| 13 | 240 mM Glycine | Turbid | Clear | 0.4 | mg/ml |
| 14 | 480 mM Glycine | Turbid | Clear | 0.2 | mg/ml |

*CFZ-API was at 50 mg/ml in DMSO, PEG400:Ethanol (1:1), Lactic Acid prior to dilution with these solubilizers in the aqueous environment.

Other excipients which include acids, amino acids and pluronics solubilized or partially solubilized CFZ-API at 2 mg/ml as seen in Table 4. Visual observations were made when pH was adjusted to approximately 3.0 with HCl, Triethanolamine (TEA), or monoethanolamine (MEA). CFZ-API recovery and stability with these excipients are still under investigation.

TABLE 4
Other Potential Solubilizers in Acidic Solution that
Were Added to Solubilize CFZ-API* at About 2 mg/ml

| | | Visual |
|---|---|---|
| | | Observation |
| No. | Solubilizers in Acidic, Water solution | at ~pH 3.0 |
| 1 | 1.3% Phenylalanine | Clear |
| 2 | 3.5% to 6.6% Arginine HCl | Clear |
| 3 | 6% Trypotophan | Clear |
| 4 | 1% Butyric Acid | Clear |
| 5 | ~0.5% to 1.6% Adipic Acid | Clear |
| 6 | ~6% N-acetyl tryptophan (in NMP) | Clear |
| 7 | ~6% N-acetyl tryptophan (in DMSO) | Turbid |
| 8 | ~6% N-acetyl tryptophan (in Benzyl Alcohol) | Turbid |
| 9 | ~6% N-acetyl tryptophan (in DMA) | Turbid |
| 10 | 5% Caprilic Acid | Turbid |
| 11 | 12% Tyrosine:Tryptophan (1:1) | Turbid |
| 12 | 7% Disodium Tyrosine | Turbid |
| 13 | 7% Pluronic F68 | Turbid |
| 14 | 7% Pluronic L64 | Slightly Turbid |
| 15 | 7.4% Pluronics L64:F68 | Slightly Turbid |

*CFZ-API was at 20 mg/ml to 50 mg/ml in NMP or DMSO, PEG400:Ethanol (1:1) prior to dilution with these solubilizers.

### Example 2. Visual Observation of the Cyclodextrin-Free CFZ-API Sample Formulation

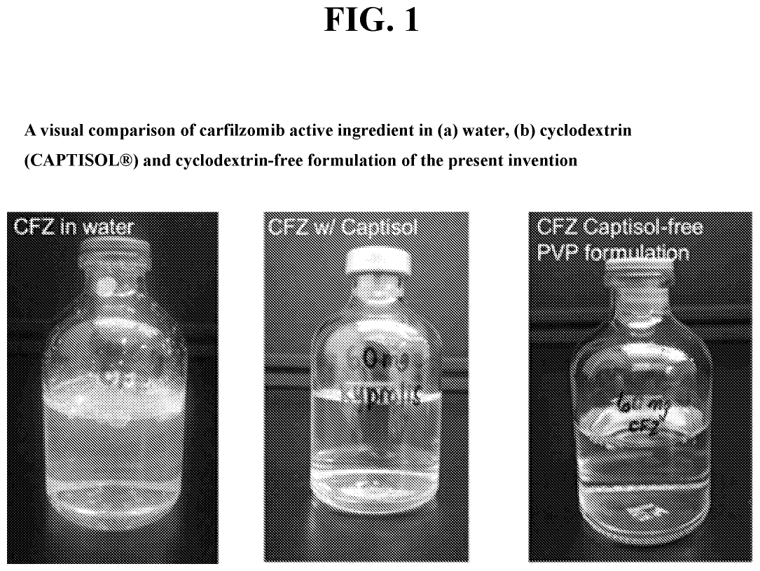

Carfilzomib (CFZ) is a proteasomal inhibitor and active ingredient of KYPROLIS®, a lyophilized drug product which is for the treatment of multiple myeloma. The current commercial formulation of KYPROLIS contains CAPTISOL®, which is a cyclodextrin used to help solubility the CFZ-API. The present invention provides a stable, cyclodextrin-free formulations for CFZ-API in an aqueous solution which is suitable for injection. FIG. 1 illustrates (a) insoluble CFZ-API in water in the presence of phosphate buffer solution (PBS) (left vial); (b) current commercial KYPROLIS formulation containing CAPTISOL (middle vial); and (c) the cyclodextrin free formulation of the invention (right vial). Each sample contains CFZ-API concentration of 2 mg/ml. The yellow tint of the cyclodextrin free polyvinylpyrillodone (PVP) formulation sample is contributed from the PVP co-solubilizing agent; however, a clear solution shows that the CFZ-API is dissolved in the formulation without cyclodextrin.

### Formulation Compositions

CFZ CYCLODEXTRIN-FREE PVP FORMULATION

| | |
|---|---|
| DMSO | 0.85% |
| PEG400 | 1.4% |
| Ethanol | 1.4% |
| Lactic acid | 0.15% |
| methane sulfonic acid | Sufficient amount to adjust pH |
| PVP 10,000 MW | 28.8% |
| Carfilzomib API | 2 mg/ml in water |

### Example 3: Solubility Modeling to Define Solvent Space

As noted in Tables 1, 2, and 5, CFZ-API can be dissolved high concentration of in water miscible solvents such as DMSO or NMP. In an effort to further understand the nature of solvation of CFZ, a solubility parameter evaluation was used to expand the solvent space of solvents that can potentially solubilize CFZ in addition to those tested in Table 1. In this approach, with the use of a software package (Hansen Solubility Parameter/HSP), solubility (originally defined by Hildebrandt as the square root of total cohesive energy) where the total parameter δ is divided into contributions from dispersive forces (δD), polar forces (δP) and hydrogen bonding (δH) forces according to the formula: δ²=δD²+δP²+δH². See https://www.hansen-solubility.com/HSP-science/basics.php. Solubility of CFZ-API in several solvents measured experimentally with results as shown in Table 1 and from previously unpublished result was used to generate the information shown in Table 5 below, with suitable solvents (CFZ solubility>200 mg/ml) identified with a score of 1 and unsuitable solvents (CFZ solubility<1 mg/ml) identified with a score of 0. A 3D solubility sphere was then obtained with the suitable solvents inside the sphere and the unsuitable solvents outside the sphere. Table 5 also shows solubility parameters obtained from the HSP software, identified as δD (dispersive), δP (polar) and δH (hydrogen bonding) forces. The Relative Energy Distance (RED) term in the table simply refers to the ratio of the distance to the center of the sphere for each solvent divided by the radius of the sphere from the center point. The distance is obtained from the well-known equation defined by Hansen, which is

$$R {a}^{2} = 4 {( δ {D}_{1} - δ {D}_{2} )}^{2} + {( δ {P}_{1} - δ {P}_{2} )}^{2} + {( δ {H}_{1} - δ {H}_{2} )}^{2} .$$

Any solvent having a RED score closer to 0 should allow for suitable solubility with CFZ. Any number greater than 1 is assumed to be an unsuitable or less suitable solvent for CFZ.

TABLE 5
Solubility Parameters for Water Miscible Solvents

| | | | | RED |
|---|---|---|---|---|
| Solvent | δD(MPa^{1/2}) | δP(MPa^{1/2}) | δH(MPa^{1/2}) | Score |
| N-Methyl-2- | 18 | 12.3 | 7.2 | 1 |
| Pyrollidone | | | | |
| N,N-Dimethyl | 16.8 | 11.5 | 9.4 | 1 |
| Acetamide | | | | |
| Dimethylsulfoxide | 18.4 | 16.4 | 10.2 | 1 |
| Ethyl Lactate | 16 | 7.63 | 12.5 | 1 |
| Benzyl Alcohol | 18.4 | 6.3 | 13.7 | 1 |
| Ethanol | 15.8 | 8.8 | 19.4 | 1 |

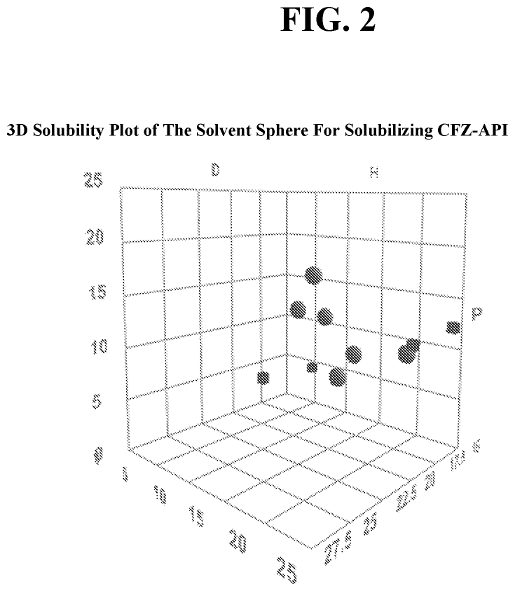

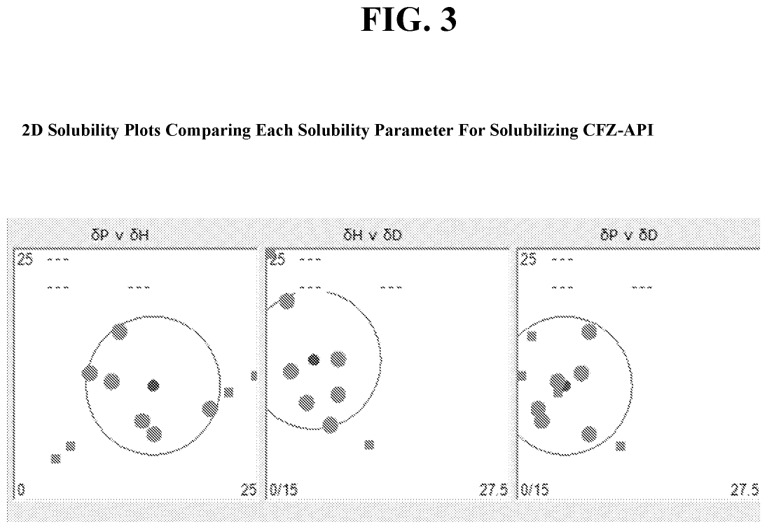

The 3D solubility plot of the solvent sphere is shown in FIG. 2. As shown, the circles refer to suitable or more suitable solvents, whereas the squares refer to unsuitable solvents for CFZ-API which was determined through this experiment. In order to learn the contribution of each of the parameters dispersive (D), polarity (P) and H bonding (H) in overall solubility, a 2D plot was drawn to compare each of the solubility parameters, δD, δP, and δH as shown in FIG. 3. FIG. 3 illustrated that based on experimental data, δP vs. δH and δH vs. δD successfully differentiated the suitable solvents from unsuitable solvents, but the correlation was weaker for δP vs. δD comparison. This suggested that the H bonding parameter was a major factor that contributed to the overall solubility pattern of CFZ, while the dispersive and polar parameters played a lesser role.

Table 5 showed that the RED score for NMP and DMSO, is close to 1, which reflected that CFZ-API would be expected to have lower solubility in these solvents. However, the inventors surprisingly found that CFZ-API were highly soluble in these solvents. This finding suggested that H bonding forces play a dominant role in determining CFZ-API solubility, and therefore solvent choices, compared to dispersive and polar forces.

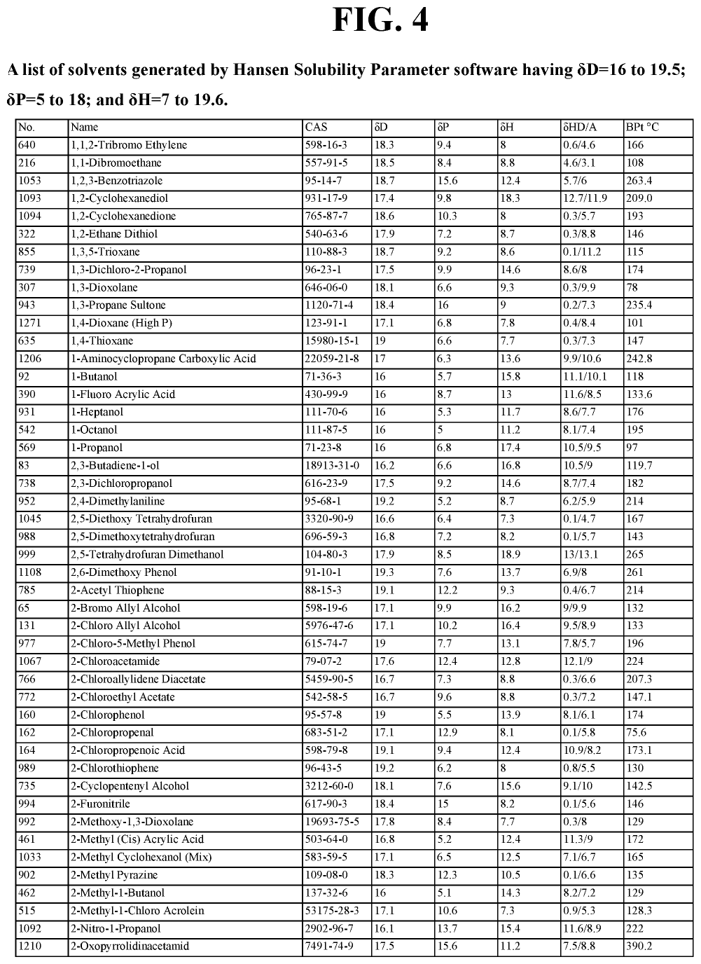

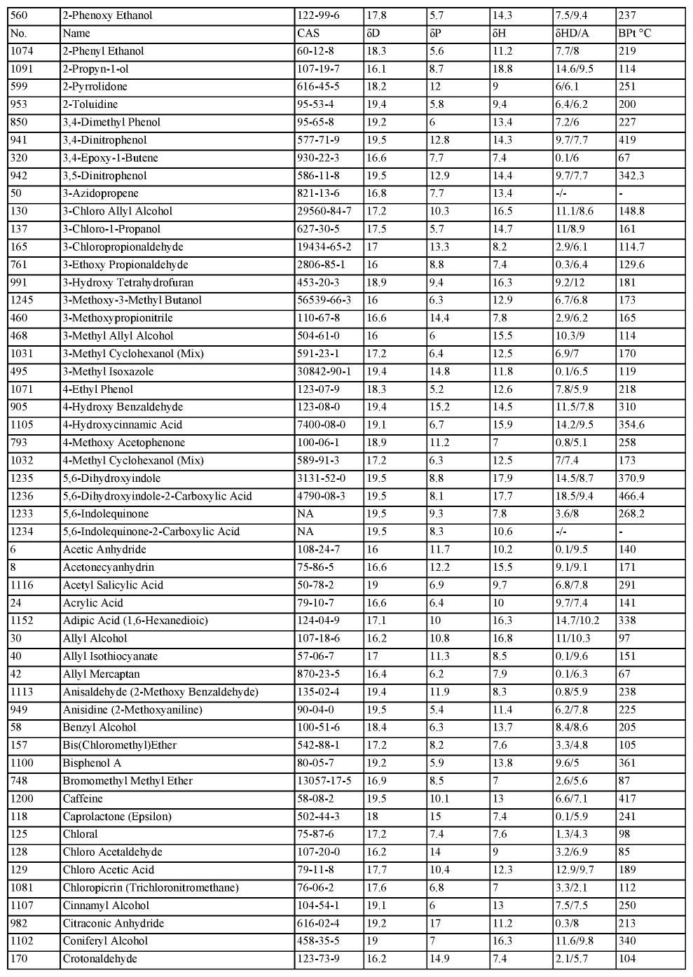

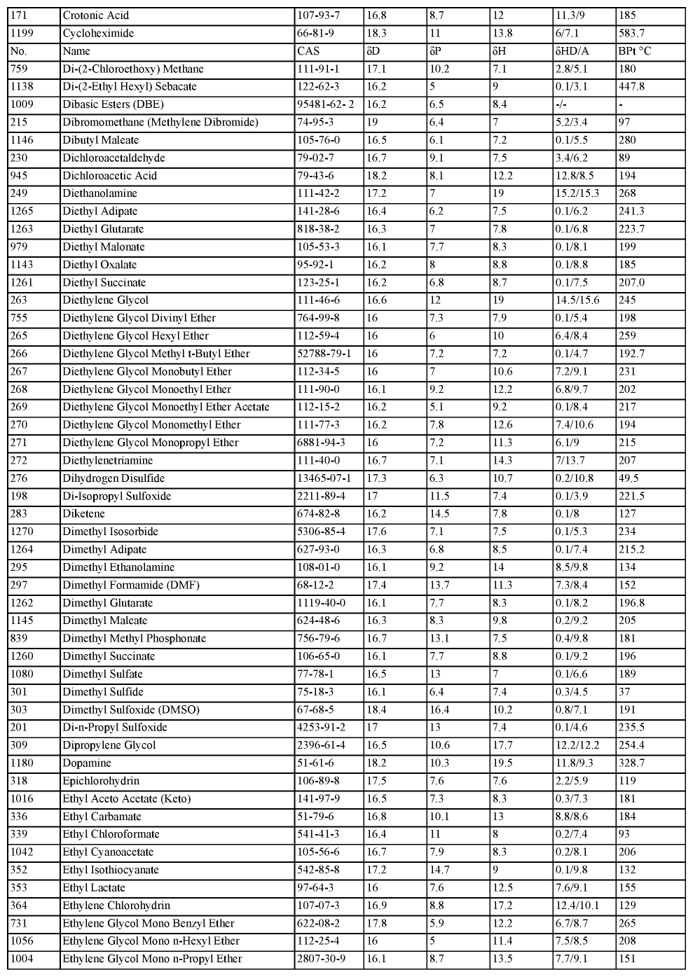

Based on the information shown in Table 5 and the plots of FIGS. 2 and 3, the present inventors can carefully select potential solvents that could be effective for CFZ, by selecting molecules within the HSP database that fall within selected solubility limits. The selected limits, based on the inventors' experimental results, were as follows: δD=16 to 19.5; δP=5 to 18; and δH=7 to 19.6. Utilizing the HSP software, a list was generated of molecules that fall within the ranges specified shown in FIG. 4. The list identifies each molecule by name, CAS number, δD, δP, δH values, δHD/A term (hydrogen bonding parameter subdivided into donor and acceptor contributions) and boiling point. Based on the details in FIG. 4, the present inventors can select varieties of suitable solvents for CFZ-API within HSP database. In addition, molecules not in HSP database but having δD, δP and δH values as specified in the range above may be expected to provide suitable or more suitable solubility profile for CFZ-API.

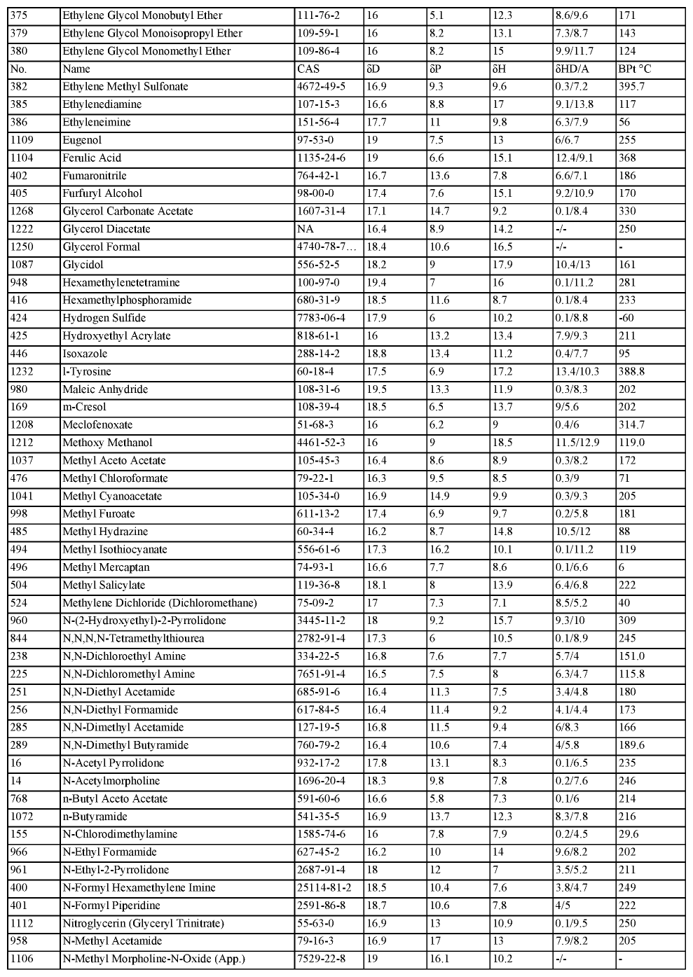

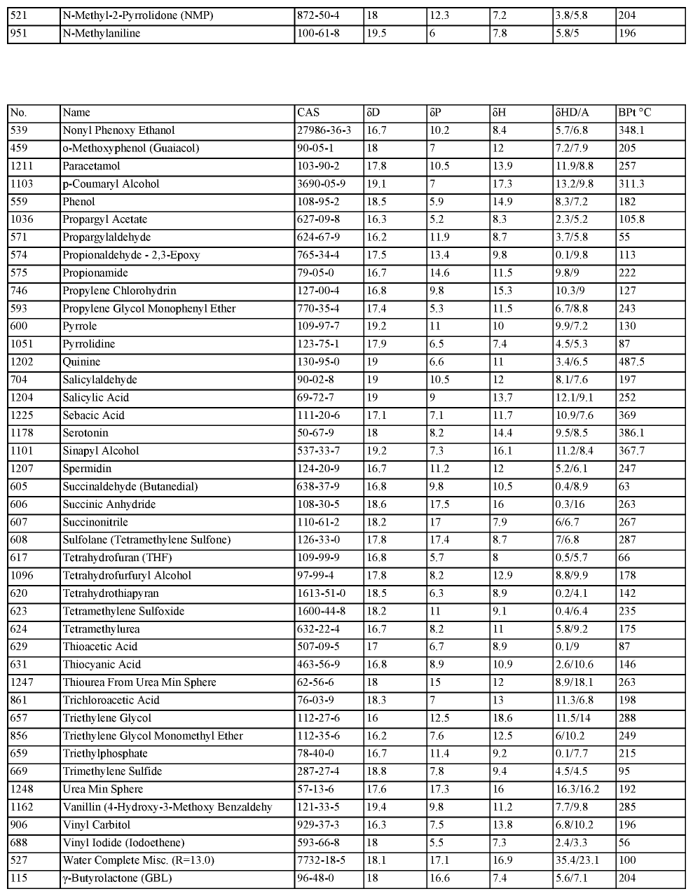

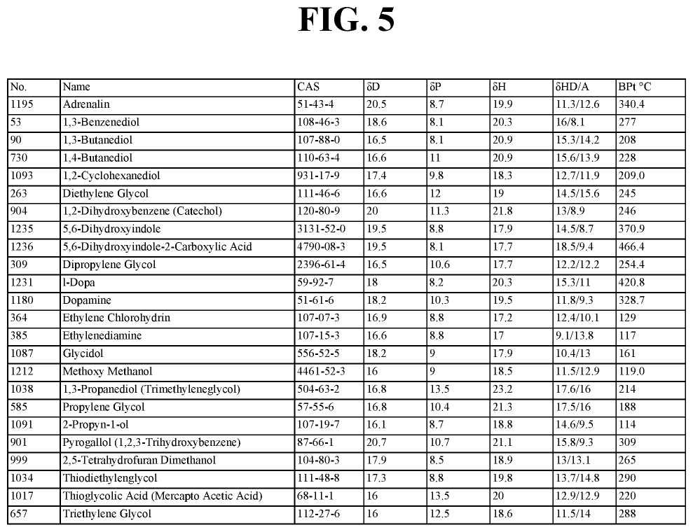

The present inventor also found that PVP helped solubilize CFZ-API upon dilution from higher concentration to lower concentration. Using the HSP software, the δD, δP and δH values for PVP were calculated to be δD=21.4, δP=11.6, and δH=21.6. Since the polymer molecular weight can have an effect on solubility, and the molecular weight is not listed in this source, a wide range for the SD, SP and SH values is not unexpected. FIG. 5 lists molecules from the solubility database that may be expected to be soluble with PVP, using the following ranges for each condition described above: Hansen source: δD=16 to 24; δP=8 to 14; and δH=17 to 24. Molecules with solubility parameters within the ranges specified here would be expected to solubilize PVP, however, since molecular weight of the PVP also affects solubility profile, identification of the suitable molecular weight of PVP suitable for dissolving CFZ-API is also useful in choosing the suitable PVP.

### Example 4: Process for Lyophilization Cycle, Bulking Agent Screening, and Results

To achieve long term stability, lyophilization of the cyclodextrin free liquid formulation of CFZ-API was considered.

### Lyophilization Process:

1 mL CFZ-API liquid formulation fill as prepared above in 3 mL Schott 1A glass vial was loaded into a VirTis Genesis 12 EL lyophilizer (TS Systems LyoStar™-3, SP Scientific, Warminster, PA). The lyophilization cycle used for screening bulking agents consisted of holding shelf temperature at 4° C. for 30 minutes then cooling the shelf to −45° C. at 0.2° C./min. The annealing step was then performed at from −45° C. to −12° C. at 0.3° C./min. The primary drying and secondary drying shelf temperatures were −25° C. and 25° C., respectively. The heating rate applied to the primary and secondary drying were 0.2° C./min and 0.1° C./min, respectively.

Table 5 describes the initial lyophilization study results of formulations containing bulking agents such as PVP 12,000 MW, mannitol, or glycine pre and post lyophilization. PVP was selected as a co-solubilizing agent candidate because of high solubility of CFZ-API in PVP pre and post lyophilization as discussed above. Since PVP are available in the market place in various molecular weight and various concentration, variety of PVP having 10,000 MW, 12,000 MW, and 17,000 MW and concentration ranges from 10% to 40% were tested. The present inventors found two solubility trends affected by PVP molecular weight sand concentration. First, CFZ-API was found to be more soluble in lower MW PVP. In this case, the 10,000 MW PVP was found to provide the highest solubility; followed by 12,000 MW PVP; and then 17,000 MW PVP. Second, a higher concentration of PVP provided higher CFZ-API solubility. The combination of these two rends revealed that to achieve the maximum CFZ-API solubility, a high concentration of a lower molecular weight PVP can be a suitable selection. It was found that 29% of 10,000 MW PVP was suitable to dissolve 2.2 mg/ml CFZ-API; while a lower concentration of 20% of 12,000 MW PVP was suitable to dissolve 2 mg/ml CFZ-API.

TABLE 5
Initial Lyophilization Screen Results

| | Pre-Lyo | | | Post-Lyo | | | |
|---|---|---|---|---|---|---|---|
| | | Osmo | | | | Osmo | |
| | [CFZ] | (mOs | | CFZ | [CFZ]/ | (mOsmo)/ | |
| BA | mg/ml | mo) | [EtOH] | cake | Recon Sln | pH | [EtOH] |
| 10% PVP | 1.2 | 681 | N/A | Poor cake | N/A | N/A | N/A |
| 12000 and | | | | | | | |
| 98 mM | | | | | | | |
| Mannitol | | | | | | | |
| 20% PVP | 1.7 | 648 | 1.38% | Better | 1.8 mg/ml/ | 360 mOsmo; | Less |
| 12000 | | | | cake; some | Water | pH 3.12 | than |
| | | | | collapsed | | | 00001% |
| 10% PVP | 1.4 | 549 | N/A | Poor cake | N/A | N/A | N/A |
| 12000 | | | | | | | |
| 192 mM | 0.9 | 645 | N/A | Collapsed | Cake | N/A osmo; | N/A |
| Mannitol | | | | cake | insoluble in | pH 3.22 | |
| | | | | | water | | |
| 98 mM | 1.0 | 549 | N/A | Poor cake | N/A | N/A | N/A |
| Mannitol | | | | | | | |
| 96 mM | 0.9 | 513 | N/A | Poor cake | N/A | N/A | N/A |
| Glycine | | | | | | | |

Table 5 Legends: BA = Bulking agent; Pre-Lyo = Pre Lyophilization; [CFZ] = CFZ solubility; Osmo = Osmolality; [EtOH] = ethanol concentration; Post-Lyo = Post-Lyophilization; CFZ cakes = CFZ Cake Appearance; and [CFZ]/Recon Sln = CFZ solubility/Reconstitution solvent.

Results from the lyophilization and bulking agent screening produced mostly poor lyophilized cakes, however two selected bulking agent provided cakes that looked better in overall drug product presentation. These two cakes (20% PVP 12,000 MW formulation and 200 mM mannitol formulation) were reconstituted with water for injection (WFI). The 20% PVP 12,000 MW formulation reconstituted to a clear solution within 5 minutes (shown in second row on Table 5). The CFZ-API recovery of 1.8 mg/ml was lower than the expected of 2 mg/ml possibly due to the other components of the powder absorbing the water, making it more dilute than theoretically measured. The observed decrease in osmolality level from pre-lyophilization to post-lyophilization was due to the ethanol evaporation, as confirmed by an ethanol quantification assay. Investigation to swap out ethanol with tert-butyl alcohol (TBA), as well as lyophilization cycle optimization may resolve the minor cake collapse observed.

### Example 5: Stability Test and Analysis

### Analytical Testing:

CFZ-API in PVP formulation prepared above was analyzed by Reserved Phase High Performance Liquid Chromatography (RP-HPLC) to accurately quantify the concentration of CFZ-API over (A) 1 day, 2 day, and 1 week storage at 2° C. to 8° C. and (B) 8 hours and 1 day storage at 25° C. Samples of three formulation solutions were compared and analyzed for stability testing, i.e., (i) KYPROLIS® (Not for Human Use/NHU); (ii) 2 mg/ml CAPTISOL® Free CFZ-API which consisted of 0.85% DMSO, 1.4% PEG400, 1.4% Ethanol, 0.15% lactic acid, and 28.8% PVP 10K; and (iii) 2 mg/ml CAPTISOL® CFZ-API which consisted of 0.85% NMP, 1.4% PEG400, 1.4% Ethanol, 0.15% lactic acid, and 28.8% PVP 10K.

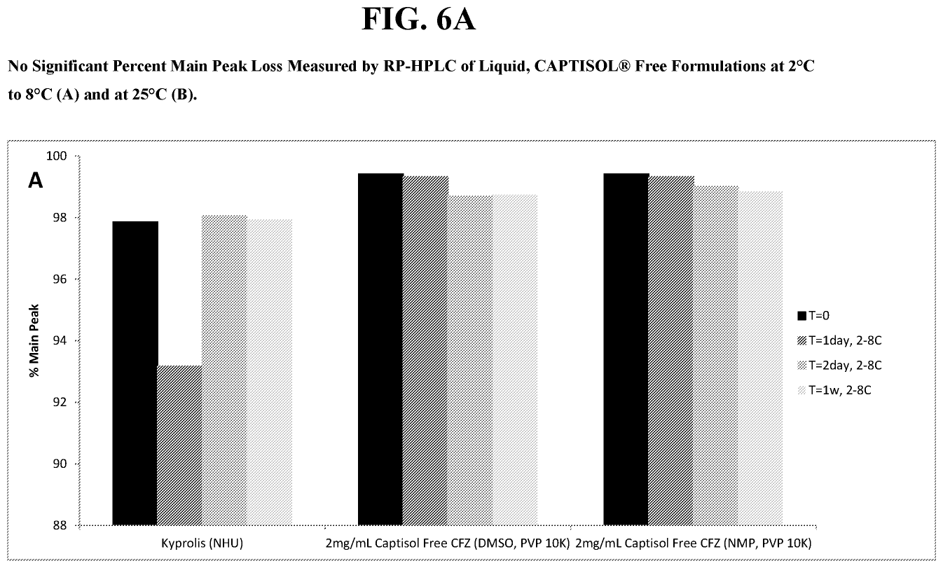

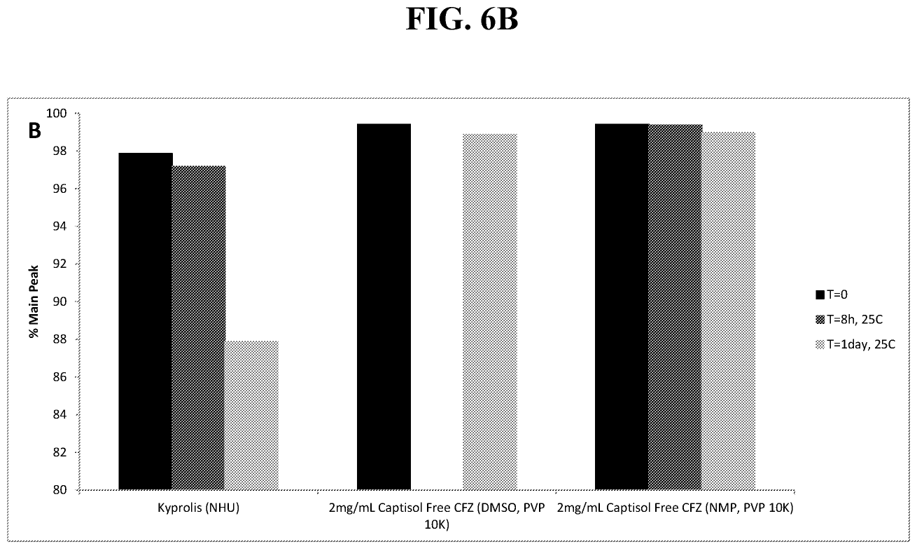

FIGS. 6A and 6B show percent main peak over storage time at 2-8° C. and 25° C., respectively. Although the CAPTISOL® Free CFZ-API in NMP had no significant loss in main peak, DMSO was found to be preferred for lower toxicity level concerns.

### Example 6—Stability Analysis of Frozen Cyclodextrin Free Carfilzomib Formulation

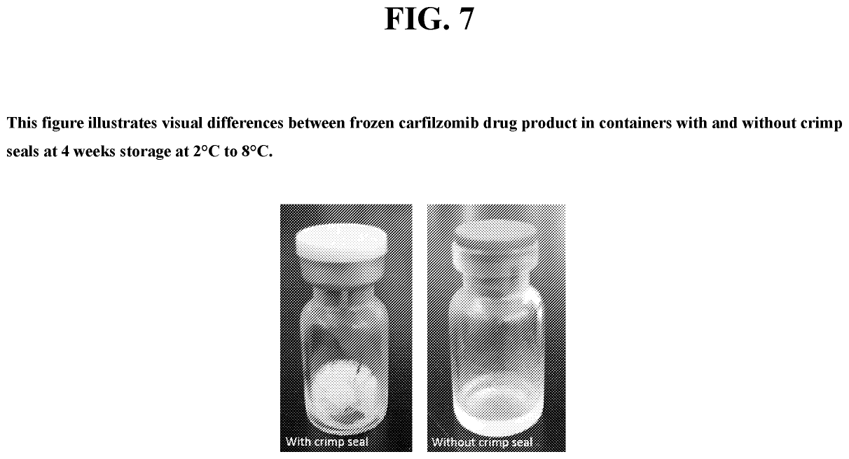

DMSO is very hygroscopic and therefore care should be taken with sample handling in low moisture conditions. In addition, moisture-free storage containers and/or devices are useful to achieve high CFZ stability and remain in frozen state at 2° C. to 8° C. Containers such as 0.5 mL microcentrifuge Eppendorf tubes and 3 cc glass Schott 1A vials with lyophilized stoppers and crimp seals were examined for CFZ in DMSO stability. FIG. 7 shows visual differences between frozen carfilzomib drug product in containers with and without crimp seals at 4 weeks storage at 2° C. to 8° C.

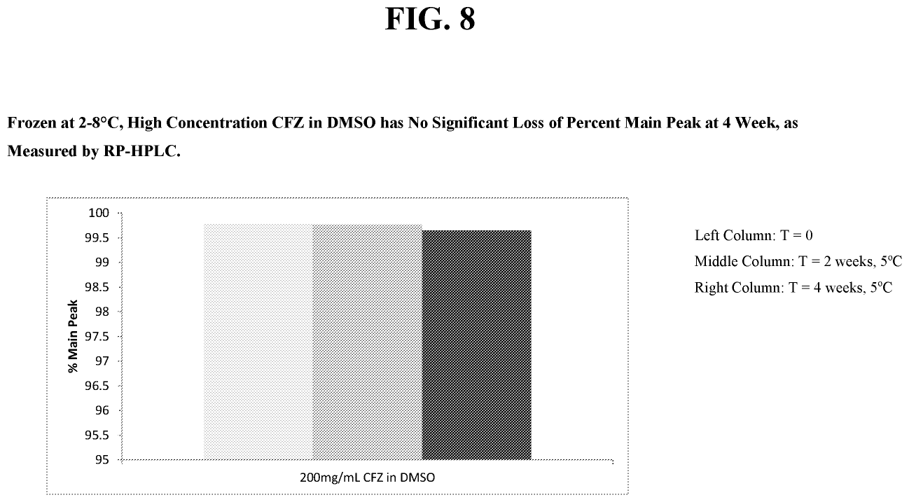

The crimp sealed container had more crystal-like frozen solid drug product compared to the frozen drug product without crimp seals. The Eppendorf tubes kept CFZ in DMSO frozen state for up to 3 weeks and then turned liquid. Drug product stability was measured by RP-HPLC, as shown in FIG. 8, for 4 weeks storage at 2° C. to 8° C. There was no significant loss in percent main peak over 4 weeks which suggested that the frozen state maintained short term stability.

### Example 7: Exploratory Single-Dose Local Tolerance Study In the Male Balb/c Mouse

Studies were conducted to measure proteasomal activity of cyclodextrin-free 2 mg/ml CFZ-API formulation of the present invention administered (A) subcutaneously and (B) intravenously to male BALB/c mice. The study was conducted at Charles River Laboratory, Inc. testing facility (Spencerville, OH). The subcutaneous and intravenous routes of exposure were selected because they are both potential routes of human exposure.

### Proteasomal Activity of Subcutaneously Administered CFZ-API to Mice

CFZ-API in PVP formulation prepared above was administered subcutaneously to mice. The test and control articles were administered to the appropriate animals via subcutaneous injection into the lower flank (caudodorsal back) area once on Day 1. The dose volume for each animal were based on the most recent body weight measurement. The animals were temporarily restrained for dose administration and were not sedated. The doses were given using a syringe with attached needle. The first day of dosing were designated as Day 1. The animal's flank area was clipped free of hair before the first dose. Care were taken during the clipping procedure to avoid abrasion of the skin. Injection site(s) (2 cm×2 cm) were delineated with an indelible marker and remarked as necessary thereafter. The result was analyzed by Reserved Phase High Performance Liquid Chromatography (RP-HPLC) to accurately quantify the proteasomal activity (Percent chymotrypsin like (% CT-L) activity) of the formulations over 5, 10, 15, and 20 hours after dosing. Samples of three formulation solutions were compared and analyzed for the pharmacodynamics testing, i.e., (i) 2 mg/ml CAPTISOL® Free CFZ-API which consisted of 0.85% DMSO, 1.4% PEG400, 1.4% Ethanol, 0.15% lactic acid, and 28.8% PVP 10K; (ii) KYPROLIS® NHU 5 mg/ml; and (iii) KYPROLIS® NHU 16.7 mg/ml.

### (B) Proteasomal Activity of Intravenously Administered CFZ-API in Mice

CFZ-API in PVP formulation prepared above was administered intravenously to mice CFZ-API in PVP formulation prepared above was administered subcutaneously to mice. The test and control articles were administered to the appropriate animals via intravenous (slow bolus) injection to the tail vein once on Day 1. The dose volume for each animal was based on the most recent body weight measurement. The animals were temporarily restrained for dose administration and were not sedated. The doses were given using a syringe with attached needle. The first day of dosing were designated as Day 1. The result was analyzed by Reserved Phase High Performance Liquid Chromatography (RP-HPLC) to accurately quantify the proteasomal activity (% CT-L activity) of the formulations over 5, 10, 15, and 20 hours after dosing. Three comparative studies were performed as follows:

| Study # | Study 1 | Study 2 | Study 3 |
|---|---|---|---|
| Formulation 1 | KYPROLIS ® NHU | KYPROLIS ® NHU 16.7 mg/ml. | 2 mg/ml CAPTISOL ® Free CFZ-API |
| | 16.7 mg/ml. | | (0.85% DMSO, 1.4% PEG400, 1.4% |
| | | | Ethanol, 0.15% lactic acid, and |
| | | | 20% PVP 12K) - Lyophilized cake |
| Formulation 2 | | 2 mg/ml CAPTISOL ® Free CFZ-API | 1.7 mg/ml CAPTISOL ® Free CFZ-API |
| | | (0.85% DMSO, 1.4% PEG400, 1.4% | (0.85% DMSO, 1.4% PEG400, 1.4% |
| | | Ethanol, 0.15% lactic acid, and | Ethanol, 0.15% lactic acid, and |
| | | 28.8% PVP 10K) - liquid formulation | 10% PVP 12K) - liquid formulation |

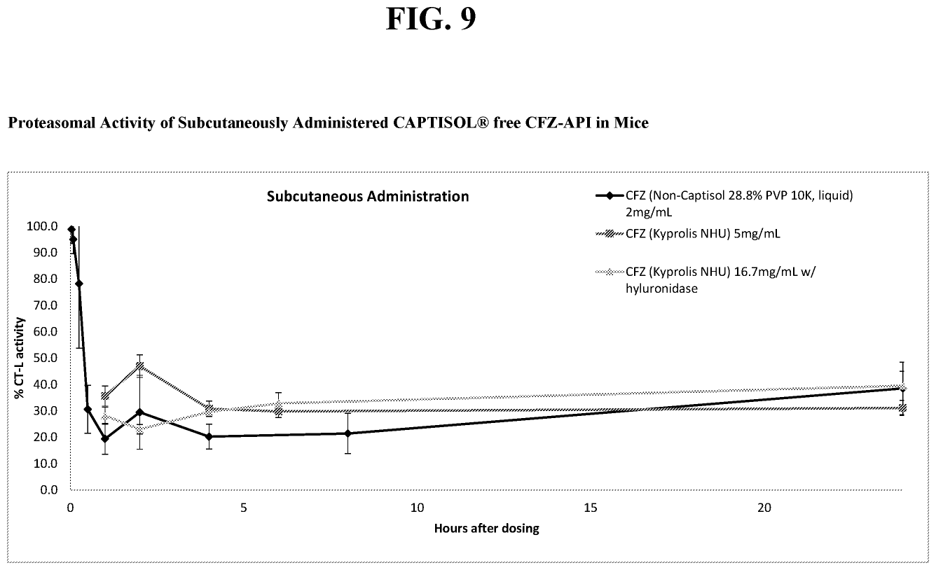

FIG. 9 showed proteasomal activity results of CAPTISOL® free CFZ-API formulation of the present invention and KYPROLIS® NHU formulation intravenously administered at 2 mg/ml in mice. For clarity, the 1.7 mg/mL CFZ-API sample was the liquid version and it was lyophilized. The lyophilized product was reconstituted to 2 mg/mL and used as a separate sample injection. A lower dosage of the cyclodextrin-free CFZ-API was shown to achieve the same efficacy as CAPTISOL® containing CFZ-API (Kyprolis NHU) in the subcutaneous administration. This decrease in dosage may translate to decrease in the toxicity exerted by CFZ-API. CAPTISOL®-free formulation that was subcutaneously administered was liquid DP with 10,000 mw PVP formulation composition. It had lower percent proteasomal activity, or higher proteasomal inhibition, than compared to KYPROLIS® NHU with and without hyaluronidase.

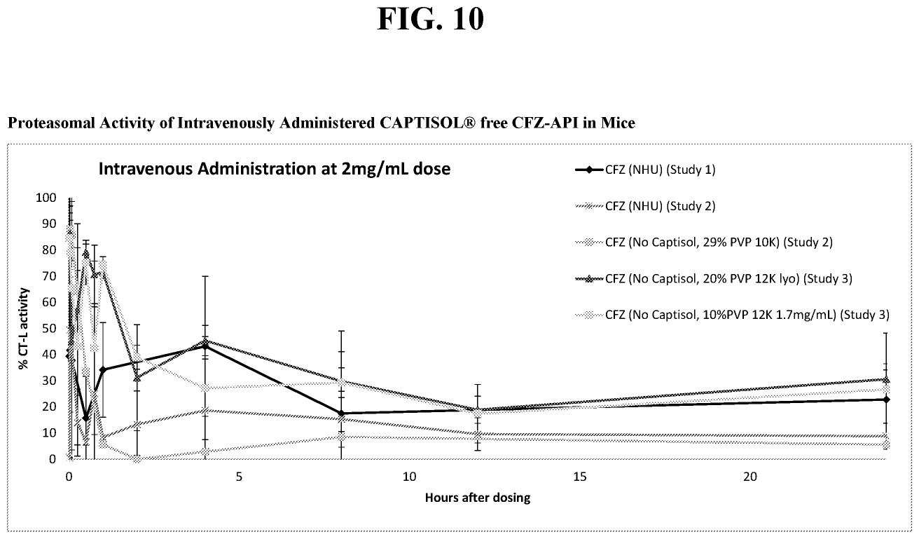

FIG. 10 shows proteasomal activity results of CAPTISOL® free CFZ-API and KYPROLIS® NHU intravenously administered at 2 mg/ml in mice. The highest proteasomal inhibition was observed in CAPTISOL® free formulation containing 28.8% PVP 10,000 mw when compared to KYPROLIS® NHU. The lyophilized PVP 12,000 MW formulation exhibited slightly lower proteasomal inhibition, however it was still in acceptable range and within standard deviation of currently marketed KYPROLIS®.

It was observed that the results from the animal studies showed significantly higher pharmacodynamics in mice from the cyclodextrin-free formulation of the present invention compared to current CAPTISOL® containing formulation. It could be hypothesized that since CAPTISOL® encapsulates CFZ-API, its bioavailability properties are compromised or less optimal than cyclodextrin-free CFZ-API. Likewise, the additives or co-solubilizers (such as PVP, DMSO, NMP, etc) may be increasing the bioavailability of CFZ-API in cyclodextrin-free formulations.

### OTHER EMBODIMENTS

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A cyclodextrin free pharmaceutical composition, comprising:
(i) carfilzomib having the chemical structure: 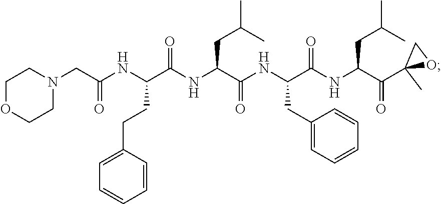 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]
or a pharmaceutically acceptable salt thereof;
(ii) a solvent system comprising a pharmaceutically acceptable solvent suitable for injection selected from the group consisting of dimethylsulfoxide, N-methyl-2-pyrrolidone, dimethylacetamide, or ethyl lactate; a co-solvent system selected from C₁₋₄ alkyl alcohol, polyethylene glycol, or combination thereof optionally in the presence of a first co-solubilizing agent; and an aqueous solution having a pH of between 2.5 to 4.5 optionally in the present of a second co-solubilizing agent;
wherein said composition is a ready-to-use injection or is obtained as a lyophilized powder or cake; and wherein the injection is administered intravenously or subcutaneously.

2. The cyclodextrin free pharmaceutical composition of claim 1, wherein the solvent system is dimethylsulfoxide, N-methyl-2-pyrrolidone, or dimethylacetimide.

3. The cyclodextrin free pharmaceutical composition of claim 1, wherein the co-solvent system is a mixture of ethanol and polyethylene glycol or a mixture of tert-butyl alcohol and polyethylene glycol optionally in the presence of the first co-solubilizing agent.

4. The cyclodextrin free pharmaceutical composition of claim 3, wherein the co-solvent system is 75% to 92% PEG400:ethanol (1:1, w/w) and in the absence of the first co-solubilizing agent.

5. The cyclodextrin free pharmaceutical composition of claim 3, wherein the co-solvent system is a mixture of ethanol and PEG400 in the presence of the first co-solubilizing agent which is selected from an acid, ester, organic salt, organic base, or a C₁₋₄ alkyl alcohol.

6. The cyclodextrin free pharmaceutical composition of claim 3, wherein the first co-solubilizing agent is an acid or an ester selected from lactic acid, maleic acid, citric acid, benzoic acid, benzene sulfonic acid, acetic acid, or sucrose cocoate.

7. The cyclodextrin free pharmaceutical composition of claim 3, wherein the co-solvent system is an organic salt selected from benzokonium chloride or protamine sulfate.

8. The cyclodextrin free pharmaceutical composition of claim 3, wherein the first co-solubilizing agent is ethanol amine or isopropyl alcohol.

9. The cyclodextrin free pharmaceutical composition of claim 3, wherein the co-solvent system is selected from the group consisting of: 75% to 92% PEG400:ethanol (1:1, w/w); 1.2% to 5% lactic acid in PEG400:ethanol; 1.2% to 5% maleic acid in PEG400:ethanol; 4.6% benzokonium chloride in PEG400:ethanol; 1% to 3.3% protamine sulfate in PEG400:ethanol; 28% to 30% HS Solutol 15 in PEG400:ethanol; 32% sucrose cocoate, PEG400:ethanol; 5% benzoic acid, PEG400:ethanol; 5% benzenesulfonic acid, PEG400:ethanol; 10% isopropyl alcohol, PEG400:ethanol; 1% to 5% citric acid in PEG400:ethanol; 1.2% to 5% acetic acid in PEG400:ethanol; or 5% ethanolamine in PEG400:ethanol.

10-34. (canceled)

35. A carfilzomib injection kit comprising:
(i) a product vial pharmaceutical composition comprising a stable lyophilized powder or cake prepared by a process comprising the steps of:
(a) dissolving said carfilzomib or a pharmaceutically acceptable salt thereof in dimethylsulfoxide, a mixture of C₁₋₄ alkyl alcohol and polyethylene glycol, and lactic acid; to form a solution wherein the concentration of the carfilzomib or said salt thereof ranges between 20 mg/ml to 50 mg/ml;
(b) diluting said carfilzomib solution with an acidic aqueous solution of water-soluble polymer and sugar mixture having a pH of between 2.5 to 4.5 to form a solution wherein the concentration of the carfilzomib or said salt thereof ranges between 1 mg/ml to 3 mg/ml; and
(c) freeze drying the solution obtained in step (b); and;
(ii) a reconstitution vial composition comprising sterilized water.
wherein said pharmaceutical composition is cyclodextrin free and the injection is administered intravenously or subcutaneously.

36. The kit of claim 35 wherein said water-soluble polymer is 1-ethenylpyrrolidin-2-one (PVP) and said sugar is mannitol or glycine or combination thereof.

37. The kit of claim 36 wherein said water-soluble polymer is PVP having a molecular weight ranges from 3,000 MW to 40,000 MW; 10,000 MW to 17,000 MW; or 10,000 MW; and said sugar is mannitol or glycine.

38-54. (canceled)

55. A process for preparation of a cyclodextrin free carfilzomib lyophilized powder or cake suitable for injection upon reconstitution comprising the steps of:
(a) dissolving the carfilzomib or a pharmaceutically acceptable salt thereof in dimethylsulfoxide, a mixture of C₁₋₄ alkyl alcohol and polyethylene glycol, and lactic acid; to form a solution wherein the concentration of the carfilzomib or said salt thereof ranges between 20 mg/ml to 50 mg/ml;
(b) diluting the carfilzomib solution with an acidic aqueous solution of water-soluble polymer and sugar mixture having a pH of between 2.5 to 4.5 to form a solution wherein the concentration of the carfilzomib or said salt thereof ranges between 1 mg/ml to 3 mg/ml; and
(c) freeze drying the solution obtained in step (b).

56. The process for preparation of a cyclodextrin free carfilzomib lyophilized powder or cake according to claim 55, wherein said water-soluble polymer is 1-ethenylpyrrolidin-2-one (PVP) and said sugar is mannitol or glycine or combination thereof.

57. The process for preparation of a cyclodextrin free carfilzomib lyophilized powder or cake according to claim 55, wherein said water-soluble polymer is PVP having a molecular weight ranges from 3,000 MW to 40,000 MW; 10,000 MW to 17,000 MW; or 10,000 MW; and said sugar is mannitol or glycine.

58-74. (canceled)

75. A method of treating multiple myeloma in a subject in need of treatment, comprising administering a therapeutically effective amount of the cyclodextrin free pharmaceutical composition of any one of claim 1, or the kit of any one of claim 35.

75-132. (canceled)
